(19) United States
(12) Patent Application Publication
(10) Pub. No.: US 2015/0374623 A1
(43) Pub. Date: Dec. 31, 2015

(54) DEAMORPHIZATION OF SPRAY-DRIED FORMULATIONS VIA SPRAY-BLENDING

(71) Applicant: NOVARTIS AG, Basel (CH)

(72) Inventors: Jeffry Weers, Belmont, CA (US); Daniel Huang, Palo Alto, CA (US); Thomas E. Tarara, Burlingame, CA (US); Danforth Miller, San Carlos, CA (US)

(21) Appl. No.: 14/768,257

(22) PCT Filed: Mar. 11, 2014
(86) PCT No.: PCT/IB2014/059632

§ 371 Date: Aug. 17, 2015

Related U.S. Application Data
(60) Provisional application No. 61/784,865, filed on Mar. 14, 2013.

(51) Int. Cl. A61K 9/00; A61M 15/00; A61K 31/40; A61K 9/16; A61K 31/4704; A61K 31/58

(52) U.S. Cl. CPC A61K 9/0075; A61K 31/4704; A61K 31/58; A61K 31/40; A61K 9/1617; A61K 9/1694; A61M 15/0045; A61M 2202/064

(57) ABSTRACT

Dry powder formulations for inhalation and their use in the treatment diseases and conditions. The formulation contains a uniform blend of a first spray-dried powder and a second spray-dried powder. The first spray-dried powder contains spray-dried particles of a therapeutically active ingredient dispersed in a pharmaceutically acceptable hydrophobic excipient. The second spray-dried powder contains spray-dried particles formed from a pharmaceutically acceptable hydrophobic excipient but are substantially free of any therapeutically active ingredient. The active ingredient in the first spray-dried powder is loaded sufficiently high to compensate for the second spray-dried powder being substantially free of any active ingredient. A process for preparing such formulations is also described.

### FIELD

This invention relates to methods for making, and compositions of, spray-dried particles prepared from an aqueous feedstock comprising a suspension of one or more active pharmaceutical ingredients. The invention further relates to organic compounds and their use as pharmaceuticals, more specifically to physically and chemically stable and substantially uniform dry powder formulations that contain one, two, three or more active ingredients. The resulting powder formulations are useful for treating a variety of diseases and conditions.

### BACKGROUND

Active pharmaceutical ingredients (APIs) that are useful for treating respiratory diseases are generally formulated for administration by inhalation with portable inhalers. Classes of portable inhalers include pressurized metered dose inhalers (pMDIs) and dry powder inhalers (DPIs).

In pharmaceutical development, there is a strong preference for crystalline APIs. Most marketed respiratory drug products, including all asthma/COPD therapeutics, are based on crystalline solids. Crystalline APIs tend toward a high level of purity and stability, particularly if the most thermodynamically stable polymorph has been identified.

Respiratory drug delivery places additional constraints on the crystalline API. First, the API often must be able to be micronized to achieve drug particles in the respirable size range from approximately 1 μm to 5 μm. The milling process can lead to a partial loss of crystallinity with the formation of amorphous or disordered material. Small amounts of such crystallographically defective material within a crystalline API may have a deleterious impact on the formulated drug product in terms of both chemical and physical stability. Most physical instability problems observed in pharmaceutical solids occur preferentially in the disordered non-crystalline regions. As a result, the API must often undergo an additional deamorphization process to increase or preserve crystallinity. Lactose blends, in particular, may require a deamorphization step to limit amorphous content in the powder particles.

Currently, most marketed inhalation products combine the micronized API with coarse lactose monohydrate to form a mixture that is inhaled by the patient. Spray drying is an alternative manufacturing process for preparing powders for inhalation.

Spray drying is a method for producing a dry powder from a liquid solution or a dispersion of particles in a liquid by drying with a hot gas. The resulting dry powders may be administered with either a DPI, or in suspension with a suitable propellant with a pMDI. Spray drying enables control of surface composition and particle morphology, factors critical in achieving good powder fluidization and dispersibility. This in turn leads to significant improvements in lung targeting and dose consistency relative to formulations based on blends of micronized API and coarse lactose monohydrate.

An advantage of spray drying is that it enables control of the physical form of the API. The API can be engineered in the spray-drying process to be either crystalline or amorphous depending on the composition of the feedstock and the spray-drying conditions. The physical form of the API in the drug product has an impact on chemical stability on storage. Some APIs are more stable as amorphous solids, while others are more stable in crystalline form. For small molecules, especially therapeutics for the treatment of asthma and chronic obstructive pulmonary disease (COPD), it is often preferred to maintain the API in crystalline form.

A method for preparing spray-dried particles incorporating crystalline API is to spray-dry a suspension of micronized API in a non-solvent liquid continuous phase. For crystalline APIs with poor solubility in water, a method is to spray-dry a suspension of API dispersed in an oil-in-water emulsion (suspension-based PULMOSPHERE™ process).

APIs for treating patients suffering from asthma and chronic obstructive pulmonary disease are highly potent with nominal doses in the range from about 5 micrograms (mcg) to 500 mcg. The minimum fill mass that can be achieved in blister receptacles for use in a dry powder inhaler (DPI) is about 500 mcg with fill masses in the range from about 1 milligram (mg) to 2 mg more practical on a high speed filling line. For capsule-based DPIs, the minimum fill mass is likely even higher, such as 2 mg to 6 mg. The high potency of asthma/COPD therapeutics and the minimum fill mass constraint places limitations on the target drug loadings in spray-dried formulations. In general, the drug loading is less than 10% w/w, more often on the order of 0.1% w/w to 5% w/w.

The high potency (low drug loadings) for spray dried formulations of asthma/COPD therapeutics places limitations on particle engineering strategies for these potent APIs. For example, in suspension-based feedstocks, where the API is dispersed as fine micronized crystals in a liquid, a low drug loading may lead to increases in the fraction of a poorly soluble crystalline drug which may dissolve in the liquid. Owing to the rapid drying kinetics in the spray-drying process (millisecond timescale), dissolved API will generally be converted into an amorphous phase in the spray dried drug product. For many APIs, the metastable amorphous phase has increased chemical degradation rates relative to the crystalline drug.

### SUMMARY

Embodiments of the present invention provide compositions which achieve the target API content in the spray-dried particles while maintaining crystallinity of the API through the spray-drying process, even when the API has a finite solubility in the liquid continuous phase of the suspension to be spray-dried. This provides deamorphization to limit amorphous content in the powder particles.

Embodiments of the present invention provide spray-dried formulations of crystalline API with reduced amorphous content, resulting in improvements in the chemical and/or physical stability of the API on storage.

Embodiments of the present invention provide compositions and methods which minimize a dissolved fraction of an API resulting in a corresponding minimization of potentially unstable amorphous API in the final product.

Embodiments of the present invention provide particles prepared by spray-drying suspensions of API, where the dose and solubility of the API are selected and/or controlled to limit dissolution of the API in the liquid phase of the feedstock.

In one aspect of the present invention, there is provided a method for reducing the dissolved fraction of an active pharmaceutical ingredient (API) in a suspension-based spray drying process, the method which comprises spray drying a feedstock comprising excipients and API at a higher drug content than is desired in the final drug product, which yields particles having a high drug content. These particles are then mixed with spray-dried vehicle particles (absent API). The resulting blend results in reduced formation of amorphous API, and as a result, improved chemical stability on storage.

In another aspect, the vehicle particles can additionally or alternatively be replaced by spray-dried particles comprising a second API and excipients, to form a fixed dose combination, of two of more actives, wherein a dissolved fraction of the first API is decreased in the fixed dose combination.

In a first aspect, the present invention relates to an engineered powder formulation for inhalation that comprises a substantially uniform blend of a first engineered powder and a second engineered powder, said first engineered powder comprising spray-dried particles that contain a crystalline therapeutically active ingredient dispersed in a pharmaceutically acceptable hydrophobic excipient, said second engineered powder comprising spray-dried particles formed from a pharmaceutically acceptable hydrophobic excipient which are substantially free of any therapeutically active ingredient, and the loading of the active ingredient in said first spray-dried powder being sufficiently high to limit dissolution of the active ingredient in the feedstock to be spray-dried.

The dry powder formulation of the present invention may contain one, two, three or more active ingredients. The additional active ingredients may be co-formulated in the first and/or second engineered powder, and/or may be formulated in a third or more engineered powder or powders. The additional active ingredients may be present in crystalline or amorphous form.

The percentage dissolved for the crystalline active ingredient in the first liquid feedstock is less than 10% w/w, preferably less than 5% w/w or 1% w/w.

In some embodiments, the first engineered powder and second engineered powder have one or more physicochemical characteristics (e.g., particle morphology, surface composition, tapped density, and primary particle size distribution) which are substantially similar. These properties are optimized to provide engineered powder blends which fluidize and disperse with little applied energy, have superior lung delivery efficiencies, and little tendency to segregate on shipping or storage.

The active ingredients can be any active pharmaceutical ingredients that are useful for treating obstructive or inflammatory airways diseases, particularly asthma and COPD. Suitable active ingredients include long acting β₂-agonists such as salmeterol, formoterol, indacaterol and salts thereof, muscarinic antagonists such as tiotropium and glycopyrronium and salts thereof, and corticosteroids including budesonide, ciclesonide, fluticasone and mometasone and salts thereof. Suitable exemplary combinations include (indacaterol maleate and glycopyrronium bromide), (indacaterol acetate and glycopyrronium bromide), (indacaterol xinafoate and glycopyrronium bromide), (indacaterol maleate and mometasone furoate), (formoterol fumarate and budesonide), (salmeterol xinafoate and fluticasone propionate), (salmeterol xinafoate and tiotropium bromide), (formoterol fumarate and tiotropium bromide), (indacaterol maleate, mometasone furoate and glycopyrronium bromide), (indacaterol acetate, mometasone furoate and glycopyrronium bromide), (indacaterol xinafoate, mometasone furoate and glycopyrronium bromide) and (formoterol fumarate, fluticasone propionate and tiotropium bromide).

In second aspect, the present invention relates to a process for preparing an inhalable dry powder formulation of spray-dried particles, the process comprising the steps of:

(a) preparing a first feedstock comprising a crystalline active ingredient dispersed in a liquid phase and a hydrophobic excipient dissolved or dispersed in a liquid phase, and spray-drying said first feedstock to provide a first engineered dry powder, wherein the drug loading of the crystalline active agent is high enough to limit dissolution in the solvent phase of the feedstock;

(b) preparing a second feedstock comprising a hydrophobic excipient dissolved or dispersed in a liquid phase, said second feedstock being substantially free of the active ingredient, and spray-drying said second feedstock to provide a second engineered dry powder substantially free of active ingredient, and;

(c) mixing the active dry powder particles and the non-active dry powder particles to provide an inhalable dry powder formulation, wherein the proportion of the non-active dry powder particles from the second feedstock is adjusted to deliver the target dose of the active ingredient in the first feedstock.

In additional embodiments, fixed dose combinations of two or more active ingredients may be prepared, where the additional active ingredients are dissolved or dispersed in either the first or second feedstock, or alternatively in an optional third or more feedstock.

In a third aspect, the present invention relates to a method for the treatment of a disease or condition which comprises administering to a subject in need thereof an effective amount of a dry powder formulation according to embodiments herein.

In a fourth aspect, the present invention relates to the use of a dry powder formulation according to embodiments herein in the manufacture of a medicament for the treatment of a disease or condition.

In a fifth aspect, the present invention relates to a dry powder formulation according to embodiments herein for use in the treatment of a disease or condition. The disease or condition may be systemic, pulmonary or both.

In a sixth aspect, the present invention relates to a method for the treatment of an obstructive or inflammatory airways disease which comprises administering to a subject in need thereof an effective amount of a dry powder formulation according to embodiments herein. The obstructive or inflammatory airways disease may comprise asthma or COPD or both.

In a seventh aspect, the present invention relates to the use of a dry powder formulation according to embodiments herein in the manufacture of a medicament for the treatment of an obstructive or inflammatory airways disease. The obstructive or inflammatory airways disease may comprise asthma or COPD or both.

In an eighth aspect, the present invention relates to a dry powder formulation according to embodiments herein for use in the treatment of an obstructive or inflammatory airways disease. The obstructive or inflammatory airways disease may comprise asthma or COPD or both.

In a ninth aspect, the present invention relates to a delivery system that comprises an inhaler that contains a dry powder formulation according to embodiments herein.

A tenth aspect of the present invention comprises any two or more of the foregoing aspects, embodiments or features.

### TERMS

Terms used in the specification have the following meanings:

“Active ingredient”, “therapeutically active ingredient”, “active agent”, “drug” or “drug substance” as used herein means the active ingredient of a pharmaceutical, also known as an active pharmaceutical ingredient (API).

“Fixed dose combination” as used herein refers to a pharmaceutical product that contains two or more active ingredients that are formulated together in a single dosage form available in certain fixed doses.

“Amorphous” as used herein refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (“glass transition”).

“Crystalline” as used herein refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (“melting point”). In the context of the present invention, a crystalline active ingredient means an active ingredient with crystallinity of greater than 85%. In certain embodiments the crystallinity is suitably greater than 90%. In other embodiments the crystallinity is suitably greater than 95%.

“Solids Concentration” refers to the concentration of active ingredient(s) and excipients dissolved or dispersed in the liquid solution or dispersion to be spray-dried.

“Drug Loading” refers to the percentage of active ingredient(s) on a mass basis in the total mass of the formulation.

“% Dissolved” refers to percentage of a crystalline active ingredient which dissolves in the liquid feedstock to be spray-dried.

“Mass median diameter” or “MMD” or “x50” as used herein means the median diameter of a plurality of particles, typically in a polydisperse particle population, i.e., consisting of a range of particle sizes. MMD values as reported herein are determined by laser diffraction (Sympatec Helos, Clausthal-Zellerfeld, Germany), unless the context indicates otherwise.

“Rugous” as used herein means having numerous wrinkles or creases, i.e., being ridged or wrinkled.

“Rugosity” as used herein is a measure of the surface roughness of an engineered particle. For the purposes of this invention, rugosity is calculated from the specific surface area obtained from BET measurements, true density obtained from helium pycnometry, and the surface to volume ratio obtained by laser diffraction (Sympatec), viz:

Rugosity=(SSA·ρₜᵣᵤₑ)/Sᵥ

where Sᵥ=6/D₃₂, where D₃₂ is the average diameter based on unit surface area. Increases in surface roughness are expected to reduce interparticle cohesive forces, and improve targeting of aerosol to the lungs. Improved lung targeting is expected to reduce interpatient variability, and levels of drug in the oropharynx and systemic circulation. In one or more embodiments, the rugosity Sᵥ is from 3 to 20, e.g., from 5 to 10.

“Emitted Dose” or “ED” as used herein refers to an indication of the delivery of dry powder from an inhaler device after an actuation or dispersion event from a powder unit. ED is defined as the ratio of the dose delivered by an inhaler device to the nominal or metered dose. The ED is an experimentally determined parameter, and may be determined using an in vitro device set up which mimics patient dosing. It is sometimes also referred to as the Delivered Dose (DD). The ED is determined using a drug specific method such as high pressure liquid chromatography.

“Emitted Powder Mass” or “EPM” as used herein refers to the mass of a powder that is delivered from an inhaler device after an actuation or dispersion event from a powder unit. The EPM is measured gravimetrically.

“Mass median aerodynamic diameter” or “MMAD” as used herein refer to the median aerodynamic size of a plurality of particles, typically in a polydisperse population. The “aerodynamic diameter” is the diameter of a unit density sphere having the same settling velocity, generally in air, as a powder and is therefore a useful way to characterize an aerosolized powder or other dispersed particle or particle formulation in terms of its settling behaviour. The aerodynamic particle size distributions (APSD) and MMAD are determined herein by cascade impaction, using a NEXT GENERATION IMPACTOR™. In general, if the particles are aerodynamically too large, fewer particles will reach the deep lung. If the particles are too small, a larger percentage of the particles may be exhaled.

“Fine particle fraction” or “FPF” as used herein means the mass of an active ingredient below a specified minimum aerodynamic size relative to the nominal dose. For example, FPF_{<3.3 μm} refers to the percentage of the nominal dose which has an aerodynamic particle size less than 3.3 μm. FPF values are determined using cascade impaction, either on an ANDERSEN™ cascade impactor, or a NEXT GENERATION IMPACTOR™ cascade impactor.

“Lung Dose” refers to the percentage of active ingredient(s) which make it past the idealized Alberta mouth-throat. Data can be expressed as a percentage of the nominal dose or the emitted dose.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

The entire disclosure of each United States patent and international patent application mentioned in this patent specification is fully incorporated by reference herein for all purposes.

### DESCRIPTION OF THE DRAWINGS

The dry powder formulation of the present invention may be described with reference to the accompanying drawings. In those drawings:

FIG. 1 is a plot showing the fraction of API dissolved in the liquid feedstock as a function of S_{API}.

FIG. 2 is a plot of drug loading vs. nominal dose for four different fill masses of API, and illustrates the impact of potency on drug loading in spray-dried formulations.

FIG. 3A is an exploded view of a nozzle assembly, and FIG. 3B is a schematic view of a multiple feedstock manifold for an atomizer nozzle known by the trademark HYDRA™.

FIG. 4 is a plot of some of the results of Example 9, namely the calculated percentage dissolved indacterol vs total impurities (S-enantiomer plus total impurities obtained via HPLC) for formulations comprising indacterol. It shows spray-blended dry powders of the present invention are more chemically stable than dry powders prepared by a conventional single particle (single nozzle) spray-drying process. The roman numerals refer to the lot numbers in Example 9.

FIG. 5 is a plot of the “lung dose” of indacaterol following aerosol administration of the marketed Onbrez drug product (standard blend) with the Breezhaler (150 mcg nominal dose). Also presented are the corresponding “lung doses” obtained for a spray-blended formulation of indacaterol delivered with the Breezhaler at nominal doses of 40 mcg, 75 mcg, and 150 mcg. The “lung dose” refers to an in-vitro measurement of the mass of powder which is delivered past the idealized Alberta mouth-throat model.

FIG. 6 is a plot of the “lung dose” of indacaterol following aerosol administration of a spray-blended formulation of indacaterol delivered with the Breezhaler at various flow rates. The “lung dose” refers to an in-vitro measurement of the mass of powder which is delivered past the idealized Alberta mouth-throat model.

FIGS. 7A-7E are photomicrographs showing particles made in accordance with embodiments of the invention.

FIG. 8 is a graph showing degradation of a prostacyclin analog compound PULMOSPHERE™ formulation as a function of the percent dissolved API in the aqueous-based feedstock to be spray-dried.

### DETAILED DESCRIPTION

Embodiments of the present invention are directed to a formulation and process to improve the chemical stability of potent APIs in a suspension-based spray-drying process by reducing the dissolved fraction of API in the suspension medium, such as the liquid. Where dissolved API is converted into amorphous APT during spray-drying and where amorphous phases often have reduced chemical stability relative to crystalline drug, embodiments of the invention form substantially crystalline drugs by limiting the amount of drug that is dissolved in the liquid during spray-drying.

The high potency (low drug loadings) for spray dried formulations of asthma/COPD therapeutics can limit particle engineering strategies for these potent APIs. For example, in suspension-based feedstocks, where the API is dispersed as fine micronized crystals in a liquid, a low drug loading may lead to increases in the fraction of a poorly soluble crystalline drug which may dissolve in the liquid. The percentage of API dissolved in a liquid feedstock is given by:

$$\% Dissolved = \frac{( {10}^{4} ) ( {S}_{API} ) ( 1 - {φ}_{PFOB} )}{( {C}_{solids} ) ( {X}_{API} )} = \frac{100 ( {S}_{API} ) ( {m}_{fill} ) ( 1 - {φ}_{PFOB} )}{( {C}_{solids} ) ( {D}_{nom} )} \qquad ( equation 1 )$$

where S_{API} is the solubility of the API (mg/ml), φ_{PFOB} is the volume fraction of pore-forming agent if present in the formulation (v/v), C_{solids} is the solids concentration in the feedstock (mg/ml), and X_{API} is the drug loading of API in the spray-dried drug product (% w/w).The drug loading is simply related to the ratio of the nominal dose Dₙₒₘ to the fill mass m_{fill}

For a 3 mg fill mass and realistic spray-drying parameters (φ_{PFOB}=0.2 and C_{solids}=30 mg/ml), the % Dissolved reduces to a simple ratio of solubility to nominal dose, viz:

$$\% Dissolved = \frac{8 ( {S}_{API} )}{( {D}_{nom} )} \qquad ( equation 2 )$$

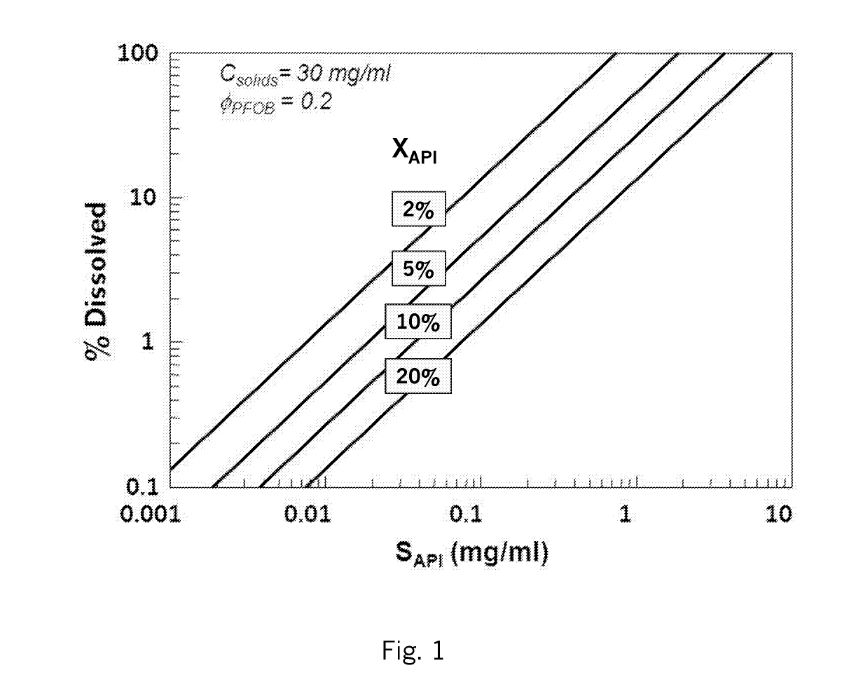

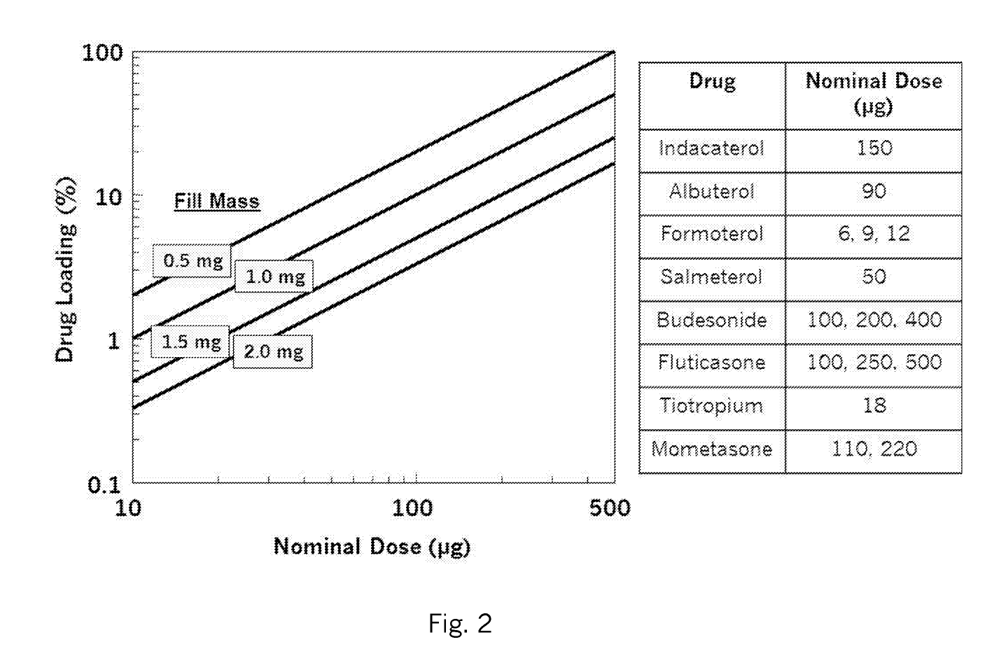

Reductions in the % dissolved can be achieved via reductions in S_{API}, or increases in C_{solids}, or X_{API}. The fraction of API dissolved in the liquid feedstock as a function of S_{API}, is plotted in FIG. 1. The various lines in FIG. 1 represent different X_{API} values. For this particular plot, it is assumed that C_{solids}=30 mg/ml and φ_{PFOB}=0.2. For X_{API}=2% to 5%, which is typical for highly potent asthma/COPD therapeutics as demonstrated in FIG. 2, that the % dissolved is 1-10%, even for an API solubility as low as 0.1 mg/ml. For a 100 mcg nominal dose, any API with a solubility of >0.16 mg/ml would result in >10% amorphous content. Hence, potent APIs with a solubility between 0.1 and 1.0 mg/ml are at risk of having significant amorphous content following spray-drying from an aqueous-based feedstock. Table 1 below further illustrates potency effects by showing a nominal dose associated with various asthma/COPD APIs.

TABLE 1

| Drug | Nominal Dose (mcg) |
|---|---|
| Indacaterol | 150 |
| Albuterol | 90 |
| Formoterol | 6, 9, 12 |
| Salmeterol | 50 |
| Budesonide | 100, 200, 400 |
| Fluticasone | 100, 250, 500 |
| Tiotropium | 18 |
| Mometosone | 110, 220, 440 |

Owing to the rapid drying kinetics in the spray-drying process (millisecond timescale), dissolved API will generally be converted into an amorphous phase in the spray dried drug product. For many APIs, the metastable amorphous phase has increased chemical degradation rates relative to the crystalline drug.

Embodiments of the process of the present invention yield dry powder formulations for inhalation comprising a blend of engineered particles wherein the particles are prepared by spray-drying an aqueous feedstock comprising a suspension of one or more APIs where the dose and solubility of the API(s) result in dissolution in the aqueous phase.

Embodiments of the process of the present invention yield dry powder formulations for inhalation comprising a blend of engineered particles, the blend containing at least one active ingredient that is suitable for treating diseases and conditions

Embodiments of the process of the present invention yield dry powder formulations for inhalation comprising a blend of engineered particles, the blend suitable for treating obstructive or inflammatory airways diseases, such as asthma and/or COPD.

In one embodiment, the dry powder formulation of the invention comprises a substantially uniform blend of a first engineered powder and a second engineered powder.

The first engineered powder comprises spray-dried particles that contain a substantially crystalline therapeutically active ingredient dispersed in a pharmaceutically acceptable hydrophobic excipient.

The drug loading of the crystalline therapeutically active ingredient in the first engineered powder is high enough to limit dissolution of the active ingredient in the liquid feedstock to be spray-dried. The percentage dissolved active ingredient in the feedstock should be less than 10% w/w, preferably less than 5% w/w, more preferably less than 1% w/w. The percentage dissolved can be measured experimentally with a drug-specific analytical method, or calculated based on the measured solubilities and feedstock composition using equation 1.

The second engineered powder comprises spray-dried particles that are formed from a pharmaceutically acceptable hydrophobic excipient and are substantially free of any therapeutically active ingredient. In some embodiments, the hydrophobic excipient of the first spray-dried powder is the same as the hydrophobic excipient of the second spray-dried powder in order to maximise blend uniformity and performance. In some embodiments the second particles contain a second drug as well as additional hydrophobic excipient to dilute the drug, e.g., concentration. The second and third drugs can be present in either crystalline or amorphous form and may be present in the same feedstock or another feedstock. The content of the second and third APIs in a fixed dose combination will be dictated by the desired nominal dose, fill mass and blend composition as discussed for the first API above. The goal for all APIs is to maintain the API as either fully crystalline or fully amorphous in the drug product.

In some embodiments, such as embodiments of a fixed dose combination comprising indacaterol as described herein, the second particles comprise a second drug as well as additional hydrophobic excipient to dilute the overall concentration of drug.

The dry powder formulation of the present invention may contain one, two, three or more active ingredients. The additional active ingredients may be co-formulated in the first or second engineered powder, or may be formulated in a third engineered powder. The additional active ingredients may be present in either crystalline or amorphous form.

A blend of two or more spray-dried powders may be prepared by physically blending the two or more powders using a mixer such as a TURBULA®. In a preferred embodiment, the particle creation and blending of the two powders occurs in a single step operation termed spray-blending. In this process, the two feedstocks are atomized into the spray-drier simultaneously with an atomizer comprising multiple twin-fluid nozzles. Under such a scenario, the mixing of particles occurs in real time as the particles are being generated leading to excellent uniformity in the blend. An exemplary spray-blending process is described in U.S. Pat. No. 8,524,279. In particular, the patent publication discloses a spray-drying process that was developed to prepare smaller particles (e.g., 0.5-50 μm) that are suitable for use in pharmaceutical products that are administered by inhalation. The process involves preparing a feedstock containing an active agent in a liquid vehicle, atomising the feedstock using a liquid atomiser to produce a droplet spray and flowing the droplet spray in a heated gas stream to evaporate the liquid vehicle to give dry particles that contain the active agent. U.S. Pat. No. 8,524,279 discloses, in general terms, that if the atomiser is provided with a plurality of feedstocks two different types of particles can be formed and blended in a single step, i.e., spray-blending. The processes and compositions of embodiments of the present invention yield a physical mixture of particles with the same or similar physicochemical properties, comprising primary particle size distributions, tapped densities, morphology and surface composition. In other words, the goal is to create a blend of particles which are substantially identical from the standpoint of interparticle cohesive forces and their resultant physical properties. Such a blend advantageously has minimal tendency to segregate on shipping or storage, and the interparticle cohesive forces will be equivalent for different drugs in a fixed dose combination, leading to equivalent aerosol performance for mono-formulations and their fixed dose combinations. Exemplary differences between lactose blends of the prior art and embodiments of spray blends of the present invention are detailed in Table 2. In the case of lactose blends, it is considered desirable to form an agglomerate between drug and carrier, so that the bulk powder properties of the drug product are improved. In embodiments of the present invention, by contrast, the composition is engineered to minimize the degree of powder agglomeration, and to make particles that readily deagglomerate with little applied energy. Hence, in embodiments of the present invention the desired bulk powder properties are built into the engineered particles themselves.

In traditional blends for inhalation, micronized drug particles (1-5 μm) are blended with inert coarse carrier particles (50-200 μm) to form an ordered mixture, in which the drug particles adhere onto the carrier particles.

TABLE 2
Comparison of traditional blends of micronized drug and
coarse lactose and spray-blends of the present invention

| | Prior Art Lactose | Embodiments of The Present |
|---|---|---|
| Attribute | Blend | Invention |
| Blend | Ordered mixtures of | Physical mixture of spray-dried |
| Characteristics | micronized API | particles with same primary |
| | (1-5 μm) and | particle size, morphology, and |
| | coarse lactose carrier | surface composition |
| Process | Micronization and | Particle creation and blending |
| | blending (two | of particles occurs in a single |
| | discreet steps) | step |
| Primary Goal | Improve powder flow | Improve chemical stability in |
| | to enable effective | suspension-based spray-drying |
| | metering of drug | processes by reducing dissolved |
| | substance | fraction |
| Lung delivery | Typically 10-30% | Target of 40-60%, with 10-20% |
| efficiencies | with 30-50% | mean variability |
| | mean variability | |
| Fixed dose | Adhesive forces for | Cohesive forces between |
| combinations | APIs with lactose | particles designed to be |
| (FDC) | will be different; | similar in physical mixture; |
| | ratio of APIs | aerosol performance of APIs |
| | delivered to the | in FDC should be equivalent |
| | lung will vary | |
| | with PIF | |

Some embodiments of the present invention comprise a process and composition comprising particles which are substantially identical from the standpoint of surface composition and morphology. In some embodiments, the feedstock and/or spray-drying process are adjusted to produce core-shell particles. In such an embodiment, the shell of the particles is comprised substantially of the hydrophobic excipient. The core of the particles contains the active ingredient(s), and additional excipients to improve chemical stability of the active ingredient(s). The particle morphology and surface composition can be “structured” or “engineered” by adjusting the feedstock composition and spray-drying conditions.

The evaporation of the volatile liquid components in an atomized droplet during spray-drying can be described as a coupled heat and mass transport problem. The difference between the vapor pressure of the liquids and their partial pressure in the gas phase is the driving force for the drying process. Two characteristic times are critical, determining the morphology of the spray-dried particles and the distribution of solid materials within the dried particles. The first is the time required for a droplet to dry, τ_{d}, and the second is the time required for materials in the atomized droplet to diffuse from the edge of the droplet to its center, R²/D. Here, R is the radius of the atomized droplet and D is the diffusion coefficient of the solutes or emulsion droplets present in the feedstock. The ratio of these two characteristic times defines the Peclet number,

$$Pe = \frac{{R}^{2}}{{τ}_{d} D} ,$$

a dimensionless mass transport number that characterizes the relative importance of the diffusion and convection processes. In the limit where drying of atomized droplets is sufficiently slow (Pe<<1), the components have an adequate time to redistribute by diffusion throughout the evaporating droplet. The end result is relatively dense particles (particle density true density of the components) with a homogenous composition. By contrast, if the drying of the atomized droplets is rapid (Pe>>1), components have insufficient time to diffuse from the surface to the center of the droplet and instead accumulate near the drying front of the atomized droplet. In such a case, low density particles with a core/shell distribution of components may occur.

The different components within the complex emulsion feedstock have different Pe (e.g., emulsion droplets versus dissolved solutes), and this drives the development of concentration gradients within the particle.

Owing to their rather large size, emulsion droplets diffuse very slowly and accumulate at the surface of the receding droplet. As the drying process continues, the evaporating front becomes a shell or crust rich in emulsion droplets enclosing the remaining solution. Eventually the remaining aqueous phase and higher boiling oil phase evaporate through the crust, leaving behind pores in place of the original liquid droplets. The particle surface is enriched in the components making up the slow diffusing emulsion droplets.

In the case of the suspension-based feedstocks described herein, the drug loadings are low and the crystalline drug particles make up a small percentage of the drug product. The drug crystals are coated with a porous layer of the hydrophobic excipient as per the Peclet discussion above. The vehicle particles, without drug, exhibit a similar surface composition as the drug-containing particles.

In general, the engineered powders of embodiments of the present invention are designed to reduce interparticle cohesive forces, by the inclusion of pores or asperities in the surface of the particles, and by the enrichment of a hydrophobic excipient at the particle interface. As such, the particles are engineered to fluidize and disperse with little applied energy, in spite of the fact that they are not blended with a coarse carrier particle. The lung delivery efficiencies are anticipated to be greater than 40% of the delivered dose. The literature shows that mean interpatient variability under such conditions will decrease to 10-20%. Degree of throat deposition can explain the variability in lung deposition of inhaled drugs. Moreover, delivery of engineered particles from a passive dry powder inhaler is expected to be largely independent of the patient's peak inspiratory flow rate (PIF). The reduction in oropharyngeal filtering and flow rate independence are expected to result in more consistent drug delivery for the engineered powders of the present invention than current marketed asthma/COPD therapeutics.

The engineered powders of the present invention will provide excellent uniformity in the emitted dose or emitted powder mass from measurement to measurement. In some embodiments, the variability is within the FDA Draft Guidance which stipulates that 90% of the measurements should be within a 20% deviation of the label claim with none outside of a 25 deviation %. In some embodiments 90% of the measurements are within a 15% deviation of the label claim, or within a 10% deviation of the label claim or mean emitted dose.

Embodiments of the present invention yield particles exhibiting a good correlation in the aerodynamic particle size distributions between two different active ingredients in a spray-blended fixed dose combination. This is assessed by direct comparison of specific stage groupings in a NEXT GENERATION IMPACTOR™ cascade impactor (NGI™). Embodiments of the present invention yield a variability in the large particle dose (stage 0 to stage 2) should be within 25%, preferably within 15% or 10%. In some embodiments variation in the fine particle dose (stage 3 to filter) is within 15%, preferably within 10% or 5%. Additionally or alternatively, in some embodiments the variation in the very fine particle fraction (stage 4 to filter) is within 15%, preferably within 10% or 5%.

Embodiments of the present invention comprise engineered particles wherein a stage grouping of stage 3 to filter provide at least 40% of a nominal dose, preferably greater than 50% or 60% of a nominal dose.

Embodiments of the present invention comprise particles which are engineered using the emulsion-based PULMOSPHERE™ dry powder manufacturing technology. The design concepts surrounding this technology are described in detail in U.S. Pat. No. 6,565,885, U.S. Pat. No. 7,871,598 and U.S. Pat. No. 7,442,388 the disclosures of which are herein incorporated in their entirety for all purposes. In particular, the method of preparing perforated microstructures for pharmaceutical applications involves spray-drying a feedstock comprising a bioactive agent, a surfactant (e.g., a phospholipid) and a blowing agent. The resulting perforated microstructures comprise the bioactive agent and the surfactant and are known as PULMOSPHERE™ particles.

Embodiments of the present invention comprise spray-blended formulations characterized by a highly uniform aerosol performance. This can be evidenced by a good correlation between gravimetric and drug specific assays for the emitted powder mass and emitted dose. In preferred embodiments the variance between the two measurements should be within 15%, preferably within 10% or 5%. The agreement between gravimetric and drug specific size distributions provides a measure of the uniformity of mixing between the two types of particles in the blend.

The presence of amorphous drug domains in crystalline micronized drugs for inhalation is generally thought to be undesirable. Amorphous domains are thermodynamically unstable, and may convert to a stable crystalline polymorph over time. The recrystallization process often results in coarsening of the micronized drug particles and decreased aerosol performance. The higher energy amorphous domains may also exhibit greater solubility, more rapid dissolution, and decreased chemical stability as compared to the crystalline drug. As a result, it is general practice to attempt to reduce the amorphous content in micronized drug particles, and companies go to great lengths to “condition” powders to reduce amorphous content. The process of the present invention minimizes the formation of amorphous domains in the active ingredient during spray-drying, by decreasing the % dissolved active ingredient in the liquid feedstock to be spray-dried.

### The Active Ingredient

The present invention is directed to formulations comprising a crystalline active ingredient with finite solubility in the liquid feedstock to be spray-dried. Embodiments of the present invention are especially useful for engineering particles comprising highly potent active ingredients with a nominal dose less than 500 mcg. Embodiments of the present invention are useful for engineering particles comprising spray-dried formulations comprising asthma and/or COPD therapeutics.

Embodiments of the present invention are useful for engineering spray-dried particles comprising one or more potent active ingredients wherein the one or more active agents is characterized by a finite solubility in the feedstock to be spray-dried, and wherein the process and formulation maintains crystallinity of the active in the resultant spray dried drug product.

Embodiments of the present invention are useful for engineering spray-dried particles comprising one or more potent active ingredients wherein the one or more active agents is characterized by a dissolved fraction as defined by Equation 1, and wherein the process and formulation maintains crystallinity of the active in the resultant spray-dried product.

The active ingredient(s) of the dry powder of the present invention can be any active pharmaceutical ingredient that is useful for treating diseases or conditions, especially treatable by pulmonary administration. The treatable disease or condition may be systemic, pulmonary, or both.

In many embodiments, the active pharmaceutical ingredient is one that is useful for treating obstructive or inflammatory airways diseases, particularly asthma and/or COPD. The active ingredient(s) may be selected, for example, from bronchodilators, anti-inflammatories, and mixtures thereof, especially long acting β₂-agonists (LABA), long acting muscarinic antagonists (LAMA), inhaled corticosteroids (ICS), dual β₂-agonist-muscarinic antagonists (MABA), PDE4 inhibitors, A_{2A} agonists, calcium blockers and mixtures thereof.

Suitable active ingredients include β₂-agonists. Suitable β₂-agonists include arformoterol (e.g., tartrate), albuterol/salbutamol (e.g., racemate or single enantiomer such as the R-enantiomer, or salt thereof especially sulfate), AZD3199, bambuterol, BI-171800, bitolterol (e.g., mesylate), carmoterol, clenbuterol, etanterol, fenoterol (e.g., racemate or single enantiomer such as the R-enantiomer, or salt thereof especially hydrobromide), flerbuterol, formoterol (e.g., racemate or single diastereomer such as the R,R-diastereomer, or salt thereof especially fumarate or fumarate dihydrate), GSK-159802, GSK-597901, GSK-678007, indacaterol (e.g., racemate or single enantiomer such as the R-enantiomer, or salt thereof especially maleate, acetate or xinafoate), abediterol, metaproterenol, milveterol (e.g., hydrochloride), naminterol, olodaterol (e.g., racemate or single enantiomer such as the R-enantiomer, or salt thereof especially hydrochloride), pirbuterol (e.g., acetate), procaterol, reproterol, salmefamol, salmeterol (e.g. racemate or single enantiomer such as the R-enantiomer, or salt thereof especially xinafoate), terbutaline (e.g., sulphate) and vilanterol (or a salt thereof especially trifenatate). In certain preferred embodiments the β₂-agonist is an ultra-long-acting β₂-agonist such as indacaterol, or potentially abediterol, milveterol, olodaterol, or vilanterol.

In some embodiments one of the active ingredients is indacaterol (i.e., (R)-5-[2-(5,6-diethyl-indan-2-ylamino)-1-hydroxyethyl]-8-hydroxy-1H-quinolin-2-one) or a salt thereof. This is a β₂-adrenoceptor agonist that has an especially long duration of action (i.e., over 24 hours) and a short onset of action (i.e., about 10 minutes). This compound is prepared by the processes described in International Patent Applications WO 2000/75114 and WO 2005/123684. It is capable of forming acid addition salts, particularly pharmaceutically acceptable acid addition salts. Pharmaceutically acceptable acid addition salts of the compound of formula I include those of inorganic acids, for example hydrofluoric acid, hydrochloric acid, hydrobromic acid or hydroiodic acid, nitric acid, sulfuric acid, phosphoric acid; and organic acids such as formic acid, acetic acid, propionic acid, butyric acid, benzoic acid, o-hydroxybenzoic acid, p-hydroxybenzoic acid, p-chlorobenzoic acid, diphenylacetic acid, triphenylacetic acid, 1-hydroxynaphthalene-2-carboxylic acid, 3-hydroxynaphthalene-2-carboxylic acid, aliphatic hydroxy acids such as lactic acid, citric acid, tartaric acid or malic acid, dicarboxylic acids such as fumaric acid, maleic acid or succinic acid, and sulfonic acids such as methanesulfonic acid or benzenesulfonic acid. These salts may be prepared from the compound by known salt-forming procedures. A preferred salt of (R)-5-[2-(5,6-diethyl-indan-2-ylamino)-1-hydroxyethyl]-8-hydroxy-1H-quinolin-2-one is the maleate salt. Another preferred salt is (R)-5-[2-(5,6-diethyl-indan-2-ylamino)-1-hydroxyethyl]-8-hydroxy-1H-quinolin-2-one acetate. Another preferred salt is (R)-5-[2-(5,6-diethyl-indan-2-ylamino)-1-hydroxyethyl]-8-hydroxy-1H-quinolin-2-one xinafoate. Other useful salts include the hydrogen succinate, fumarate, hippurate, mesylate, hydrogen sulphate, hydrogen tartrate, hydrogen chloride, hydrogen bromide, formate, esylate, tosylate, glycolate and hydrogen malonate salts, which, like the acetate and xinafoate salts, are disclosed in International Patent Application WO 2008/000839 together with methods of their respective preparation.

Suitable active ingredients include muscarinic antagonists or antimuscarinics. Suitable muscarinic antagonists include aclidinium (e.g., bromide), BEA-2180 (e.g., bromide), CHF-5407, darifenacin (e.g., bromide), darotropium (e.g., bromide), glycopyrrolate (e.g., racemate or single enantiomer, or salt thereof especially bromide), dexpirronium (e.g., bromide), iGSK-202405, umeclidinium, GSK-656398, ipratropium (e.g., bromide), LAS35201, otilonium (e.g., bromide), oxitropium (e.g., bromide), oxybutynin, PF-3715455, pirenzepine, revatropate (e.g., hydrobromide), solifenacin (e.g., succinate), TD-4208, terodiline, tiotropium (e.g., bromide), tolterodine (e.g., tartrate), and trospium (e.g., chloride). In certain preferred embodiments the muscarinic antagonists is long-acting muscarinic antagonist such as darotropium bromide, umeclidinium, glycopyrrolate or tiotropium bromide.

In some embodiments one of the active ingredients is a glycopyrronium salt. Glycopyrronium salts include glycopyrronium bromide, also known as glycopyrrolate, which is known to be an effective antimuscarinic agent. More specifically it inhibits acetyl choline binding to M3 muscarinic receptors thereby inhibiting bronchoconstriction. Glycopyrrolate is a quaternary ammonium salt. Suitable counter ions are pharmaceutically acceptable counter ions including, for example, fluoride, chloride, bromide, iodide, nitrate, sulfate, phosphate, formate, acetate, trifluoroacetate, propionate, butyrate, lactate, citrate, tartrate, malate, maleate, succinate, benzoate, p-chlorobenzoate, diphenyl-acetate or triphenylacetate, o-hydroxybenzoate, p-hydroxybenzoate, 1-hydroxynaphthalene-2-carboxylate, 3-hydroxynaphthalene-2-carboxylate, methanesulfonate and benzenesulfonate. Glycopyrrolate can be prepared using the procedures described in U.S. Pat. No. 2,956,062. It has two stereogenic centers and hence exists in four isomeric forms, namely (3R,2′R)-, (3S,2′R)-, (3R,2′S)- and (3S,2′S)-3-[(cyclopentyl-hydroxyphenyl-acetyl)oxy]-1,1-dimethylpyrrolidinium bromide, as described in United States patent specifications U.S. Pat. No. 6,307,060 and U.S. Pat. No. 6,613,795. When the drug substance of the dry powder formulation is glycopyrrolate, it can be one or more of these isomeric forms, especially the 3S,2′R isomer, the 3R,2′R isomer or the 2S,3′R isomer, thus including single enantiomers, mixtures of diastereomers, or racemates, especially (3S,2′R/3R,2′S)-3-[(cyclopentyl-hydroxy-phenylacetyl)oxy]-1,1-dimethylpyrrolidinium bromide. R,R-glycopyrrolate is also known as dexpirronium.

Suitable active ingredients include bifunctional active ingredients such as dual β₂-agonists-muscarinic antagonists. Suitable dual β₂-agonists-muscarinic antagonists include GSK-961081 (e.g., succinate).

In some embodiments the active ingredient(s) of the dry powder of the present invention can be any active pharmaceutical ingredient that is useful for treating pulmonary arterial hypertension and/or related diseases. Suitable active ingredients include any having efficacy against such disease(s) such as signalling molecules, platelet aggregation inhibitors and vasodilators. In some embodiments the active comprises a prostacyclin analog.

Suitable active ingredients include steroids, for example corticosteroids. Suitable steroids include budesonide, beclamethasone (e.g., dipropionate), butixocort (e.g., propionate), CHF5188, ciclesonide, dexamethasone, flunisolide, fluticasone (e.g., propionate or furoate), GSK-685698, GSK-870086, LAS40369, methyl prednisolone, mometasone (e.g., furoate), prednisolone, rofleponide, and triamcinolone (e.g., acetonide). In certain preferred embodiments the steroid is long-acting corticosteroids such as budesonide, ciclesonide, fluticasone or mometasone.

In one embodiment one of the active ingredients is mometasone (i.e., (11β, 16α)-9,21-dichloro-17-[(2-furanylcarbonyl)oxy]-11-hydroxy-16-methylpregna-1,4-diene-3,20-dione, alternatively designated 9α,21-dichloro-16α-methyl-1,4-pregnadiene-11β,17α-diol-3,20-dione 17-(2′-furoate)) or a salt thereof, for example mometasone furoate and mometasone furoate monohydrate. Mometasone furoate and its preparation are described in U.S. Pat. No. 4,472,393. Its use in the treatment of asthma is described in U.S. Pat. No. 5,889,015. Its use in the treatment of other respiratory diseases is described in U.S. Pat. No. 5,889,015, U.S. Pat. No. 6,057,307, U.S. Pat. No. 6,057,581, U.S. Pat. No. 6,677,322, U.S. Pat. No. 6,677,323 and U.S. Pat. No. 6,365,581.

Pharmaceutically acceptable esters, acetals, and salts of the above therapeutics are contemplated. The determination of the appropriate esters, acetals, or salt form is driven by the duration of action and tolerability/safety data. As well, API selection may be important from the standpoint of selecting therapeutics with the appropriate physical properties (e.g., solubility) to achieve the embodiments of the present invention.

### Combinations

The dry powder formulation of the present invention can contain two, three, four or more therapeutically active ingredients that are useful for treating diseases and conditions.

In some embodiments, the diseases or conditions comprise obstructive or inflammatory airways diseases, particularly asthma and COPD. Particularly preferred fixed dose combinations include combinations of APIs from the following families: LABA/ICS, LABA/LAMA, LABA/LAMA/ICS, and MABA/ICS.

Suitable combinations include those that contain a β₂-agonist and a corticosteroid. Exemplary embodiments of combinations are shown by the parantheticals: (carmoterol and budesonide), (formoterol and beclomethasone), (formoterol fumarate and budesonide), (formoterol fumarate dihydrate and mometasone furoate), (formoterol fumarate and ciclesonide), (indacaterol maleate and mometasone furoate), (indacaterol acetate and mometasone furoate), (indacaterol xinafoate and mometasone furoate), (milveterol hydrochloride and fluticasone), (olodaterol hydrochloride and fluticasone furoate), (olodaterol hydrochloride and mometasone furoate), (salmeterol xinafoate and fluticasone propionate), (vilanterol trifenatate and fluticasone furoate), and(vilanterol trifenatate and mometasone furoate); a β₂-agonist and a muscarinic antagonist, for example (formoterol and aclidinium bromide), (indacaterol and darotropium), (indacaterol maleate and glycopyrrolate); (indacaterol acetate and glycopyrrolate); (indacaterol xinafoate and glycopyrrolate); (indacaterol maleate and umeclidinium), (milveterol hydrochloride and glycopyrrolate), (milveterol hydrochloride and tiotropium bromide), olodaterol hydrochloride and glycopyrrolate), (olodaterol hydrochloride and tiotropium bromide), (salmeterol xinafoate and tiotropium bromide), (vilanterol trifenatate and darotropium), (vilanterol trifenatate and glycopyrrolate), (vilanterol trifenatate and umeclidinium), and (vilanterol trifenatate and tiotropium bromide); and a muscarinic antagonist and a corticosteroid, for example (glycopyrrolate and mometasone furoate), and (glycopyrrolate and ciclesonide); or a dual β₂-agonist-muscarinic antagonist and a corticosteroid, for example (GSK-961081 succinate and mometasone furoate), (GSK-961081 succinate and mometasone furoate monohydrate), and (GSK-961081 succinate and ciclesonide). It should be noted that virtually any combinations are possible, including combinations between actives described in parentheticals, and with others.

Some embodiments of the present invention comprise spray-dried particles comprising two active ingredients. Some embodiments of the present invention comprise spray-dried particles comprising three active ingredients.

Suitable triple combinations include those that contain a β₂-agonist, a muscarinic antagonist and a corticosteroid, for example (salmeterol xinafoate, fluticasone propionate and tiotropium bromide), (indacaterol maleate, mometasone furoate and glycopyrrolate), (indacaterol acetate, mometasone furoate and glycopyrrolate) and (indacaterol xinafoate, mometasone furoate and glycopyrrolate).

Some embodiments of the present invention comprise spray-dried particles comprising more than three active ingredients.

### Excipients

The minimum fill mass of fine powder that can be reasonably filled commercially on a high speed filling line with a relative standard deviation of less than 3% is about 0.5 mg. In contrast, the required lung dose of active ingredients may be as low as 0.01 mg, and routinely is about 0.2 mg or less. Hence, significant quantities of excipients are usually required.

In some embodiments, the dry powder formulation of the present invention contains a pharmaceutically acceptable hydrophobic excipient.

The hydrophobic excipient may take various forms that will depend at least to some extent on the composition and intended use of the dry powder formulation. Suitable pharmaceutically acceptable hydrophobic excipients may, in general, be selected from the group consisting of long-chain phospholipids, hydrophobic amino acids and peptides, and long chain fatty acid soaps.

In some embodiments, formulations of the present invention comprise a first and a second engineered powder. In such embodiments, the first engineered powder of the dry powder formulation comprises spray-dried particles that contain a therapeutically active ingredient dispersed in a pharmaceutically acceptable hydrophobic excipient. The second engineered powder of the dry powder formulation comprises spray-dried particles that are formed from a pharmaceutically acceptable hydrophobic excipient (and do not contain any therapeutically active ingredient).

In some embodiments, the hydrophobic excipient of the first spray-dried powder is the same as the hydrophobic excipient of the second spray-dried powder in order to maximise blend uniformity and performance. In some embodiments, the excipient of the first spray-dried powder is different from the excipient of the second spray-dried powder.

The content of the hydrophobic excipient in the formulation may be determined from the nominal dose of the API and the fill mass (X_{exc}=(1−X_{API})=1−(Dₙₒₘ/m_{fill})).

By control of the formulation and process, it is possible for the surface of the first spray-dried particles to be comprised primarily of the hydrophobic excipient. Surface concentrations may be greater than 70%, such as greater than 75% or 80% or 85%. In some embodiments the surface is comprised of greater than 90% hydrophobic excipient, or greater than 95% or 98% or 99% hydrophobic excipient. For potent APIs it is not uncommon for the surface to be comprised of more than 95% hydrophobic excipient.

In some embodiments the hydrophobic excipient facilitates development of a rugous particle morphology. This means the particle morphology is porous, wrinkled and creased rather than smooth. This means the interior and/or the exterior surface of the inhalable medicament particles are at least in part rugous. This rugosity is useful for providing dose consistency and drug targeting by improving powder fluidization and dispersibility. Increases in particle rugosity result in decreases in inter-particle cohesive forces as a result of an inability of the particles to approach to within van der Waals contact. The decreases in cohesive forces are sufficient to dramatically improve powder fluidization and dispersion in ensembles of rugous particles.

The rugosity of the particles may be increased by using a pore-forming agent, such as perflubron, during their manufacture, or by controlling the formulation and/or process to produce rugous particles.

Phospholipids from both natural and synthetic sources may be used in varying amounts. When phospholipids are present, the amount is typically sufficient to provide a porous coating matrix of phospholipids. If present, phospholipid content generally ranges from about 40 to 99% w/w of the medicament, for example 70% to 90% w/w of the medicament. The high percentage of excipient is also driven by the high potency and therefore typically small doses of the active ingredients. Given that no carrier particle is present in the spray-dried particles, the excipients also serve as bulking agents in the formulation, enabling effective delivery of low dose therapeutics. In some embodiments, it is also desirable to keep the drug loading low to ensure that the particle properties are controlled by the surface composition and morphology of the particles. This enables comparable physical stability and aerosol performance between mono and combination particles to be achieved, even for blends of engineered particles with comparable surface composition and particle morphology.

Generally compatible phospholipids comprise those having a gel to liquid crystal phase transition greater than about 40° C., such as greater than 60° C., or greater than about 80° C. The incorporated phospholipids may be relatively long chain (e.g., C₁₆-C₂₂) saturated phospholipids. Exemplary phospholipids useful in the disclosed stabilized preparations include, but are not limited to, phosphatidylcholines, such as dipalmitoylphosphatidylcholine (DPPC), distearoylphosphatidylcholine (DSPC), and hydrogenated egg or soy phosphatidylcholines (e.g., E-100-3, S-100-3, available from Lipoid KG, Ludwigshafen, Germany). Natural phospholipids are preferably hydrogenated, with a low iodine value (<10).

The phospholipids may optionally be combined with cholesterol to modify the fluidity of the phospholipid acyl chains.

The long-chain phospholipids may optionally be combined with a divalent metal ion (e.g., calcium, magnesium). Such a divalent metal ion acts to decrease headgroup hydration, thereby increasing the phospholipid gel to liquid crystal phase transition, and the wettability of the powders on lung lining fluid. The molar ratio of polyvalent cation to phospholipid may be at least about 0.05:1, such as about 0.05:1 to 0.5:1. In one or more embodiments, a molar ratio of polyvalent cation:phospholipid is 0.5:1. While not intending to be bound by theory, it is believed that the divalent metal ion binds to the phosphate groups on the zwitterionic phosphatidylcholine headgroup, displacing water molecules in the process. Molar ratios of metal ion to phospholipid in excess of 0.5 may result in free metal ion not bound to the phosphate groups. This can significantly increase the hygroscopicity of the resulting dry powder, and is not preferred. When the polyvalent metal ion is calcium, it may be in the form of calcium chloride. Although metal ions, such, as calcium, are often included with phospholipids, none is required, and their use can be problematic when other ions are present in the formulation (e.g., phosphate, which may precipitate the calcium ions as calcium phosphate). When compatibility issues occur, there may be benefit in using Mg⁺⁺ salts, as they typically have Kₛₚ values which are three to four orders of magnitude higher than Ca⁺⁺ salts.

The hydrophobic excipient may also comprise long chain fatty acid soaps. The alkyl chain length is generally 14-22 carbons in length with saturated alkyl chains preferred. The fatty acid soaps may utilize monovalent (e.g., Na⁺, K⁺) or divalent counterions (e.g., Ca⁺⁺, Mg⁺⁺). Particularly preferred fatty acid soaps are sodium stearate and magnesium stearate. The solubility of fatty acid soaps may be increased above the Krafft point. Potassium salts of fatty acids generally have the lowest Krafft point temperature, and greater aqueous solubility at a given temperature. Calcium salts are expected to have the lowest solubility. The hydrophobic fatty acid soaps provide a wax-like coating on the particles. The proposed loadings in the spray-dried particles are similar to the phospholipids detailed previously.

The hydrophobic excipient may also comprise hydrophobic amino acids, peptides, or proteins. Particularly preferred are the amino acid leucine, and its oligomers dileucine and trileucine. Proteins, such as, human serum albumin are also contemplated. Trileucine is particularly preferred, as its solubility profile and other physicochemical properties (e.g., surface activity, log P) facilitate creation of core-shell particles, where trileucine controls the surface properties and morphology of the resulting particles.

Other excipients contemplated include salts, buffers, and glass-forming agents. Of particular significance to the formulation of spray blends to prevent dissolution of API in the feedstock is the addition of the conjugate base of the acid used to form the salt of the API. For example, for indacaterol maleate, the conjugate base is sodium maleate. When indacaterol maleate is placed in water, an equilibrium is established between indacaterol maleate, indacaterol free base and sodium maleate. Addition of sodium maleate shifts the equilibrium towards the salt form, thereby lowering the solubility of the salt, and reducing amorphous content in the spray-dried powder. This is often referred to as the common ion effect. The common ion may also serve as a buffer and glass-forming excipient in the formulation.

Traditional glass-forming agents (e.g., carbohydrates, amino acids, buffers) are also contemplated. Particularly preferred are sucrose, trehalose, mannitol, and sodium citrate).

### Formulation

Embodiments of the present invention provide a dry powder formulation that comprises a chemically stable and substantially uniform blend of spray-dried particles.

Embodiments of the present invention comprise engineered particles comprising a porous or rugous surface. Such particles exhibit reduced interparticle cohesive forces compared to micronized drug crystals of a comparable primary particle size. This leads to improvements in powder fluidization and dispersibility relative to ordered mixtures of micronized drug and coarse lactose.

Embodiments of dry powder formulations of the present invention may comprise 0.1 to 50% w/w of active ingredients, or 0.1 to 40% w/w of active ingredients, or 0.1% to 30% w/w of active ingredient(s), such as 0.5% to 10% w/w, or 2% to 5% w/w.

In some embodiments, crystalline active ingredients are micronized. The MMD (x50) of the micronized active ingredients should be less than 3.0 μm, preferably less than 2.0 μm, or 1.0 μm. The x90 should be less than 7 μm, preferably less than 5 μm, or 3 μm.

The dry powder formulation of the present invention may comprise one or more excipients in addition to the aforementioned hydrophobic excipient. Such additional excipients are sometimes referred to herein as “additives.”

In one or more embodiments of the dry powder formulation of the present invention, the formulation may additionally include additives to further enhance the stability or biocompatibility of the formulation. For example, various salts, buffers, chelators, bulking agents, common ions, glass forming excipients, and taste masking agents are contemplated. Other additives suitable for use in compositions according to the invention are listed in “Remington: The Science & Practice of Pharmacy,” 19^{th} ed., Williams & Williams, (1995), and in the “Physician's Desk Reference,” 52^{nd} ed., Medical Economics, Montvale, N.J. (1998) both of which are incorporated herein by reference in their entireties.

In some embodiments, particularly those comprising active agent and phospholipid, a hydrophobic excipient makes up the balance of the formulation. That is it serves as both a surface modifier and a bulking agent in the formulation. In such embodiments, the content of the hydrophobic excipient in the dry powder formulation of the present invention is greater than 70% w/w of the composition, often greater than 90% w/w, or 95% w/w, or 99% w/w of the composition of a given particle. The hydrophobic excipient loading can be as high as 99.9% w/w.

The use of hydrophobic excipients such as trileucine may be limited by their solubility in the liquid feedstock. Typically, the content of trileucine in an engineered powder is less than 30% w/w, more often on the order of 10% w/w to 20% w/w. Owing to its limited solubility in water and its surface activity, trileucine is an excellent shell former. As a result trileucine is generally mixed with a bulking agent, which is present in the core of the particle with the crystalline active ingredient. Leucine may also be used as a shell forming excipient and embodiments of the invention may comprise particles with achieve leucine concentrations of up to about 50%. Fatty acid soaps behave similarly to leucine and trileucine and are thus suitable surface modifiers.

The bulking agents may be glass-forming excipients with a high glass transition temperature (>80° C.). Embodiments of the present invention may comprise glass forming agents such as sucrose, trehalose, lactose, mannitol and sodium citrate. These bulking agents can additionally or alternatively aid in stabilizing any amorphous active ingredient present in the formulation.

In certain preferred embodiments the hydrophobic excipient comprises greater than 70% of the particle interface as measured by Electron Spectroscopy for Chemical Analysis (ESCA, also known as X-ray photoelectron spectroscopy or XPS), preferably greater than 90% or 95%.

In some embodiments the particles of the dry powder formulation of the preset invention suitably have a mass median diameter (MMD) of between 1 and 5 microns, for example of between 1.5 and 4 microns.

In some embodiments the particles of the dry powder formulation of the invention suitably have a mass median aerodynamic diameter (MMAD) of between 1 and 5 microns, for example of between 1 and 3 microns.

In some embodiments the particles of the dry powder formulation of the invention suitably have a rugosity of greater than 1.5, for example from 1.5 to 20, 3 to 15, or 5 to 10.

In some embodiments in order to minimize interpatient variability in lung deposition, the particles of the dry powder formulation of the invention suitably have a fine particle fraction, expressed as a percentage of the nominal dose<3.3 μm (FPF_{<3.3 μm}) of greater than 40%, preferably greater than 50%, but especially greater than 60%. Lung deposition as high as 50-60% of the nominal dose (60-80% of the delivered dose) is contemplated.

In some embodiments the fine particle dose of particles of the dry powder formulation of the invention having a diameter less than 4.7 μm (i.e., FPF_{<4.7 μm}) is suitably greater than 50%, for example of between 40% and 90%, especially of between 50% and 80%. This minimizes interpatient variability associated with oropharyngeal filtering.

When the formulation of the present invention contains two active ingredients the differences in FPF_{<3.3 μm} for the two active ingredients are suitably less than 15%, preferably less than 5%.

In some embodiments, the “lung dose” as measured using the idealized Alberta mouth-throat is greater than 50% of the emitted dose, for example between 50% and 90%, especially between 50% and 80% of the emitted dose.

### Process

The present invention provides a process for preparing dry powder formulations for inhalation, comprising a blend of spray-dried particles, the blend containing at least one active ingredient. Embodiments of the present invention provide a process for preparing dry powder formulations for inhalation, comprising a blend of spray-dried particles, the blend containing at least one active ingredient that is suitable for treating obstructive or inflammatory airways diseases, particularly asthma and/or COPD.

Spray drying confers advantages in producing engineered particles for inhalation such as the ability to rapidly produce a dry powder and control of particle attributes including size, morphology, density, and surface composition. The drying process is very rapid (in the order of milliseconds). As a result most active ingredients which are dissolved in the liquid phase precipitate as amorphous solids, as they do not have sufficient time to crystallize.

Spray-drying comprises four unit operations: feedstock preparation, atomization of the feedstock to produce micron-sized droplets, drying of the droplets in a hot gas, and collection of the dried particles with a bag-house or cyclone separator.

Embodiments of the process of the present invention comprise three steps, however in some embodiments two or even all three of these steps can be carried out substantially simultaneously, so in practice the process can in fact be considered as a single step process. Solely for the purposes of describing the process of the present invention the three steps will be described separately, but such description is not intended to limit to a three step process.

In embodiments of a first step of the process of the invention active dry powder particles are prepared by preparing a first feedstock and spray-drying the feedstock to provide active dry powder particles.

The first feedstock comprises at least one active ingredient and a pharmaceutically acceptable hydrophobic excipient dispersed in a liquid feedstock or vehicle. The first feedstock is provided with a loading of the active ingredient that is sufficiently high to reduce the fraction of active ingredient that dissolves in the liquid feedstock to be spray-dried.

The choice of liquid feedstock (or vehicle) depends on the physicochemical properties of the active ingredients. Useful liquids from which to make a selection include water, ethanol, ethanol/water, acetone, dichloromethane, dimethylsulfoxide, and other Class 3 solvents as defined in ICH Q3C Guidelines, for example ICH Topic Q3C (R4) Impurities: Guideline for Residual Solvents (European Medicines Agency reference CPMP/ICH/283/95 of February 2009).

In some embodiments, the active ingredient is poorly soluble in water so the preferred liquid is water. When the active ingredient comprises indacaterol or a salt thereof the liquid is suitably water.

The solubility of the active ingredient in the feedstock to be spray-dried can be decreased by decreasing the temperature of the feedstock. As a rule of thumb, solubility decreases two-fold with each 10° C. decrease in temperature. Hence, going from room temperature to refrigerated conditions would be expected to decrease solubility about 4-fold.

In some instances, the addition of salts which “salt out” the active ingredient may be utilized to further expand the range of insoluble active ingredients that can be prepared within the context of the invention. It may also be possible to modify the pH or add common ions for active ingredients with ionisable groups to limit solubility according to Le Chatelier's Principle. The nature of the salt can be utilized to modify the physicochemical properties, in particular the solubility, of the active ingredient.

Once the solubility of the crystalline active ingredient is known, the required drug loading to achieve a target % dissolved in the feedstock can be calculated using equation 1.

In some embodiments, the % dissolved crystalline active ingredient is less than 10% w/w, preferably less than 5% w/w or 1% w/w.

The particle size distribution of the insoluble crystalline active ingredient is important in achieving uniformity within atomized droplets during spray-drying. Embodiments of the present invention provide that the x₅₀ (median diameter) should be less than 3.0 μm, preferably less than 2.0 μm, or even 1.0 μm. In some embodiments the crystalline insoluble particles may be nano sized, i.e., x50<1000 nm or 200 nm. The x90 should be less than 7 μm, preferably less than 5 μm, preferably less than 4 μm or even 3 μm. For nanoparticles, the x90 should be less than about 1000 nm.

In embodiments where the dry powder will contain two or more of the active ingredients that are substantially insoluble in water, it is often preferred that they have a similar primary particle size distribution, so that the aerodynamic particle size distribution and pattern of lung deposition are similar for the active ingredients in the mono formulations.

In embodiments comprising feedstocks comprising oil-in-water emulsions, the dispersed oil phase serves as a pore-forming agent to increase particle porosity and rugosity in the spray-dried drug product. Suitable pore-forming agents include various fluorinated oils including perfluorooctyl bromide (perflubron), perfluorodecalin, and perfluorooctyl ethane. The emulsion droplets may be stabilized by a monolayer of a long-chain phospholipid, which serves as the hydrophobic excipient in the spray-dried particles.

In embodiments of the invention, an emulsion may be prepared by first dispersing the hydrophobic excipient in hot distilled water (e.g., 70° C.) using a suitable high shear mechanical mixer (e.g., ULTRA-TURRAX T-25 mixer) at 8000 rpm for 2 to 5 minutes. If the hydrophobic excipient is a phospholipid, a divalent metal, e.g., calcium chloride may be added to decrease headgroup hydration. The fluorocarbon is then added drop-wise while mixing. The resulting fluorocarbon-in-water emulsion may then be processed using a high pressure homogenizer to reduce the particle size. Typically, the emulsion is processed for two to five discrete passes at 8,000 to 20,000 psi to produce droplets with a median diameter less than 600 nm. The active ingredient is added into the continuous phase of the emulsion and mixed and/or homogenized until it has dispersed and a suspension has been formed. Additional excipients/additives are dissolved in the continuous phase of the emulsion.

In some embodiments, the feedstock is aqueous-based, however inhalable dry powder formulations of the present invention may also be prepared using organic solvents or bisolvent systems. Ethanol/water systems are especially useful as a means to control the solubility of one or more of the materials comprising the particle. Solvent-based systems are especially useful for formulations comprising hydrophobic excipients, e.g., trileucine, and/or leucine, which are dissolved in the liquid feedstock.

It is important to control the moisture content of the drug product. For drugs which are not hydrates the moisture content in the powder is preferably less than 5%, more typically less than 3%, or even 2% w/w. Moisture content must be high enough, however, to ensure that the powder does not exhibit significant electrostatic attractive forces. The moisture content in the spray-dried powders may be determined by Karl Fischer titrimetry.

In some embodiments, the feedstock is sprayed into a current of warm filtered air that evaporates the solvent and conveys the dried product to a collector. The spent air is then exhausted with the solvent. Operating conditions of the spray-dryer such as inlet and outlet temperature, feed rate, atomization pressure, flow rate of the drying air, and nozzle configuration can be adjusted in order to produce the required particle size, moisture content, and production yield of the resulting dry particles. The selection of appropriate apparatus and processing conditions are within the purview of a skilled artisan in view of the teachings herein and may be accomplished without undue experimentation. Exemplary settings for a NIRO® PSD-1® scale dryer are as follows: an air inlet temperature between about 80° C. and about 200° C., such as between 110° C. and 170° C.; an air outlet between about 40° C. to about 120° C., such as about 60° C. and 100° C.; a liquid feed rate between about 30 g/min to about 120 g/min, such as about 50 g/min to 100 g/min; total air flow of about 140 scfm to about 230 scfm, such as about 160 scfm to 210 scfm; and an atomization air flow rate between about 30 scfm and about 90 scfm, such as about 40 scfm to 80 scfm. The solids content in the spray-drying feedstock will typically be in the range from 0.5% w/v (5 mg/ml) to 10% w/v (100 mg/ml), such as 1.0% w/v to 5.0% w/v. The settings will, of course, vary depending on the scale and type of equipment used, and the nature of the solvent system employed. In any event, the use of these and similar methods allow formation of particles with diameters appropriate for aerosol deposition into the lung.

On drying, a skin of the hydrophobic phospholipid forms on the surface of the particles. The water soluble drug and excipients diffuse throughout the atomized droplets. Eventually, the oil phase evaporates leaving behind pores in the spray-dried particles, and a rugous particle morphology. The nature of the particle surface and morphology will be controlled by controlling the solubility and diffusivity of the components within the feedstock. Surface active hydrophobic excipients (e.g., trileucine, phospholipids, fatty acid soaps) may be concentrated at the interface, improving powder fluidization and dispersibility, while also driving increased surface roughness for the particles.

In embodiments comprising feedstocks where the excipients are all dissolved in the feedstock, core-shell coatings on the dispersed active ingredient(s) are driven by differences in the physical properties of the dissolved solutes.

A pore-forming agent may be added in order to increase the surface rugosity of the particles. This improves the fluidization and dispersibility characteristics of the particles.

In embodiments of a second step of the process of the invention non-active dry powder particles are prepared from a second feedstock and that feedstock is spray-dried to provide the non-active dry powder particles. The second feedstock comprises a pharmaceutically acceptable hydrophobic excipient, and is preferably substantially free of the active ingredient.

The particles may optionally contain an additional additive to bulk the composition. While this may not be needed when the emulsion-based feedstocks are utilized, additional bulking agents are needed for excipients like trileucine which have limited solubility in an aqueous or ethanolic feedstock. Preferred bulking agents are carbohydrates such as sucrose, trehalose, sugar alcohols like mannitol, or salts or buffers.

A ratio of the non-active containing dry powder particles to active-containing particles will be determined by the drug loading required for the active containing dry powder particles to limit dissolution of the crystalline drug in the liquid feedstock to be spray-dried. In some embodiments, the non-active particles in essence serve the role of a “filler” to achieve the desired drug loading required to deliver a therapeutic dose of the API at an acceptable fill mass in the powder receptacle.

The hydrophobic excipient used to prepare the second feedstock may be the same hydrophobic excipient used to prepare the first feedstock or may be a different hydrophobic excipient. In embodiments where the same hydrophobic excipient is used for both the active dry powder particles formed in the first step and the non-active dry powder particles formed in the second step, the resulting dry powder formulation of the invention is often characterized by substantially identical physicochemical properties, which yields the desired blend uniformity.

The choice of liquid depends on the physicochemical properties of the active ingredients. Useful liquids from which to make a selection include water, ethanol, ethanol/water, acetone, dichloromethane, dimethylsulfoxide, and other Class 3 solvents as defined in ICH Q3C Guidelines, for example ICH Topic Q3C (R4) Impurities: Guideline for Residual Solvents (European Medicines Agency reference CPMP/ICH/283/95 of February 2009).

Any spray-drying step and/or all of the spray-drying steps may be carried out using conventional equipment used to prepare spray dried particles for use in pharmaceuticals that are administered by inhalation. Commercially available spray-dryers include those manufactured by Büchi Ltd. and Niro Corp.

As discussed previously for the particles comprising a crystalline active ingredient, the nature of the particle surface and morphology will be controlled by controlling the solubility and diffusivity of the components within the feedstock. Surface active hydrophobic excipients (e.g., trileucine, phospholipids, fatty acid soaps) may be concentrated at the interface, improving powder fluidization and dispersibility, while also driving increased surface roughness for the particles.

For embodiments comprising fixed dose combinations comprising two or more active ingredients, the active ingredients may be dissolved or dispersed in either the first or second feedstock, or additionally or alternatively, in a third feedstock. The additional active ingredients may be formulated in either crystalline or amorphous form.

In embodiments of a third step of the process of the invention the active dry powder particles and the non-active dry powder particles are mixed or blended to provide the inhalable dry powder formulation of the invention.

The active dry powder particles prepared in the first step can be mixed with the non-active dry powder particles prepared in the second step using conventional mixing equipment.

In some embodiments the first, second and third steps are conveniently carried out in a single step particle creation and blending process, “spray-blending”. In this process, the active dry powder particles are ejected from one or more spray dryer nozzles and mix with the non-active dry powder particles that are ejected from one or more other spray dryer nozzles located in close proximity. This can be readily achieved using a multi-headed atomizer fed by individual feedstocks. Such a multi-headed atomizer is disclosed in U.S. Pat. No. 8,524,279, Snyder et al.

Relative to conventional mechanical blending operations, spray-blending eliminates the need for intermediate storage, reduces the risk of product contamination and/or product loss, and reduces capital equipment costs thereby reducing production time and costs. Moreover, the spray blending process reduces the potential for triboelectric charging, which can be problematic in traditional blending operations.

Blend uniformity may be analysed using the active ingredient(s) in the spray blended formulations post-filling into a foil-foil blister. In this regard, the content values should at minimum meet current regulatory guidelines for content uniformity, which state that the relative standard deviation (RSD) should be less than or equal to 6%. In some embodiments herein, the content uniformity RSD should be less than 5%, or less than 4% or less than 3% or less than 2% at least one of or two of or each of the beginning, middle, and end of the batch. In some embodiments of the process and formulation of the present invention, the uniformity of the content values is maintained during shipping and on storage of the drug product over a period of at least two years.

### Use in Therapy

Embodiments of the present invention provide a method for the treatment of an obstructive or inflammatory airways disease, especially asthma and chronic obstructive pulmonary disease, the method which comprises administering to a subject in need thereof an effective amount of the aforementioned dry powder formulation.

In one or more embodiments, a method of treatment comprises administering to a subject a dry powder formulation comprising three actives (“trombo”) comprising about 0.5-3% w/w indacaterol maleate, about 0.5-3% w/w mometasone furoate, about 0.5-3% w/w glycopyrronium bromide, about 89-98% DSPC plus CaCl₂, and about 0.1-1% w/w maleic acid (as buffer).

In one or more embodiments, a method of treatment comprises administering to a subject a dry powder formulation comprising two actives (“combo”) comprising about 0.5-3% w/w indacaterol maleate, about 0.5-3% w/w mometasone furoate, about 93-99% w/w DSPC plus CaCl₂, and about 0.1-1% w/w maleic acid (as buffer).

In one or more embodiments, a method of treatment comprises administering to a subject a dry powder formulation comprising two actives (“combo”): comprising about 0.5-3% w/w indacaterol maleate, about 0.5-3% w/w glycopyrronium bromide, about 93-99% DSPC plus CaCl₂, and about 0.1-1% w/w maleic acid (as buffer).

The present invention also relates to the use of the aforementioned dry powder formulation in the manufacture of a medicament for the treatment of an obstructive or inflammatory airways disease, especially asthma and chronic obstructive pulmonary disease.

The present invention also provides the aforementioned dry powder formulation for use in the treatment of an obstructive or inflammatory airways disease, especially asthma and chronic obstructive pulmonary disease.

Treatment of a disease or condition in accordance with the invention may be symptomatic or prophylactic treatment or both.

Exemplary obstructive or inflammatory airways diseases to which the present invention is applicable include asthma of whatever type or genesis including both intrinsic (non-allergic) asthma and extrinsic (allergic) asthma. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, for example anti-inflammatory (e.g., corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g., between the hours of about 4 to 6 am, i.e., at a time normally substantially distant form any previously administered symptomatic asthma therapy.

Other obstructive or inflammatory airways diseases and conditions to which the present invention is applicable include acute/adult respiratory distress syndrome (ARDS), chronic obstructive pulmonary or airways disease (COPD or COAD), including chronic bronchitis, or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further obstructive or inflammatory airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis. Also contemplated is bronchiectasis associated with cystic fibrosis, and non-CF bronchiectasis.

Embodiments of the dry powder formulation of the present invention are especially useful for treating Asthma, COPD or both.

Exemplary systemic diseases and conditions to which the present invention is applicable include, but are not limited to Pulmonary Arterial Hypertension.

### Unit Dosage Form

The present invention also provides a unit dosage form, comprising a container containing a dry powder formulation of the present invention.

In one embodiment, the present invention is directed to a unit dosage form, comprising a container containing a dry powder formulation of three actives (“trombo”): comprising about 0.5-3% w/w indacaterol maleate, about 0.5-3% w/w mometasone furoate, about 0.5-3% w/w glycopyrronium bromide, about 89-98% DSPC plus CaCl₂, and about 0.1-1% w/w maleic acid (as buffer). In embodiments of the invention, the unit dosage form comprises a fill mass of from 0.5 mg to 10 mg.

In one embodiment, the present invention is directed to a unit dosage form, comprising a container containing a dry powder formulation of two actives (“combo”): comprising about 0.5-3% w/w indacaterol maleate, about 0.5-3% w/w mometasone furoate, about 93-99% w/w DSPC plus CaCl₂, and about 0.1-1% w/w maleic acid (as buffer). In embodiments of the invention, the unit dosage form comprises a fill mass of from 0.5 mg to 10 mg.

In one embodiment, the present invention is directed to a unit dosage form, comprising a container containing a dry powder formulation of two actives (“combo”) comprising about 0.5-3% w/w indacaterol maleate, about 0.5-3% w/w glycopyrronium bromide, about 93-99% DSPC plus CaCl₂, and about 0.1-1% w/w maleic acid (as buffer). In embodiments of the invention, the unit dosage form comprises a fill mass of from 0.5 mg to 10 mg.

Examples of containers include, but are not limited to, capsules, blisters, or container closure systems made of metal, polymer (e.g., plastic, elastomer), glass, or the like.

The container may be inserted into an aerosolization device. The container may be of a suitable shape, size, and material to contain the dry powder formulation and to provide the dry powder formulation in a usable condition. For example, the capsule or blister may comprise a wall which comprises a material that does not adversely react with the dry powder formulation. In addition, the wall may comprise a material that allows the capsule to be opened to allow the dry powder formulation to be aerosolized. In one or more versions, the wall comprises one or more of gelatin, hydroxypropylmethyl-cellulose (HPMC), polyethyleneglycol-compounded HPMC, hydroxypropylcellulose, agar, aluminium foil, or the like. For current marketed asthma/COPD therapeutics, the fill mass in the container is in the range from 0.5 mg to 10 mg, preferably in the range from 1 mg to 4 mg.

The use of foil-foil blisters are also contemplated. The selection of appropriate foils for the blister is within the purview of a skilled artisan in view of the teachings herein. The nature of the foils utilized will be driven by the moisture permeability of the seal, and the ability of the material to be formed into a blister of the appropriate size and shape. In one embodiment, the powder is loaded into foil-foil blisters with a fill mass of between 0.5 and 10 mg, preferably 1.0 mg to 4.0 mg.

### Delivery System

The present invention also provides a delivery system, comprising an inhaler and a dry powder formulation of the invention.

In one embodiment, the present invention is directed to a delivery system, comprising a dry powder inhaler and a dry powder formulation for inhalation that comprises a substantially uniform blend of a first engineered powder and a second engineered powder, said first engineered powder comprising spray-dried particles that contain a therapeutically active ingredient dispersed in a pharmaceutically acceptable hydrophobic excipient, said second engineered powder comprising spray-dried particles that are formed from a pharmaceutically acceptable hydrophobic excipient and are substantially free of any therapeutically active ingredient, and the loading of the active ingredient in said first spray-dried powder being sufficiently high to limit dissolution of the active ingredient in the feedstock to be spray-dried.

### Inhalers

Suitable inhalers include dry powder inhaler (DPIs). Some such inhalers include unit dose inhalers, where the dry powder is stored in a capsule or blister, and the patient loads one or more of the capsules or blisters into the device prior to use. Other multi-dose dry powder inhalers include those where the dose is pre-packaged in foil-foil blisters, for example in a cartridge, strip or wheel.

Preferred dry powder inhalers include multi-dose dry powder inhalers such as the DISKUS™ (GSK, described in U.S. Pat. No. 6,536,427), DISKHALER™ (GSK, described in WO 97/25086), GEMINI™ (GSK, described in WO 05/14089), GYROHALER™ (Vectura, described in WO 05/37353), PROHALER™ (Valois, described in WO 03/77979) and TWISTHALER™ (Merck, described in WO 93/00123, WO 94/14492 and WO 97/30743) inhalers.

Preferred single dose dry powder inhalers include the AEROLIZER™ (Novartis, described in U.S. Pat. No. 3,991,761) and BREEZHALER™ (Novartis, described in US Patent Application Publication 2007/0295332 (Ziegler et al.). Other suitable single-dose inhalers include those described in U.S. Pat. Nos. 8,069,851 and 7,559,325.

Preferred unit dose blister inhalers, which some patients find easier and more convenient to use to deliver medicaments requiring once daily administration, include the inhaler described by in US Patent Application Publication US2010/0108058 to Glusker et al.

Particularly preferred inhalers are multi-dose dry powder inhalers where the energy for fluidizing and dispersing the powder is supplied by the patient (i.e., “passive” MD-DPIs). The powders of the present invention fluidize and disperse effectively at low peak inspiratory flow rates (PIF). As a result, the small changes in powder dispersion with PIF observed effectively balance the increases in inertial impaction which occur with increases in PIF, leading to flow rate independent lung deposition. The absence of flow rate dependence observed for powders of the present invention, drives reductions in overall interpatient variability. Suitable blister-based passive multi-dose inhalers include the DISKUS™ (GSK), GYROHALER™ (Vectura), DISKHALER™ (GSK), GEMINI™ (GSK), and PROHALER™ (Valois) devices.

Some patients may prefer to use an “active” multi-dose dry powder inhaler where the energy for fluidizing and dispersing the powder is supplied by the inhaler. Suitable such inhalers include pressurizable dry powder inhalers, as disclosed, for example in WO 96/09085, WO 00/072904, WO 00/021594 and WO 01/043530, and ASPIRAIR™ (Vectura) inhalers. Other active devices may include those available from MicroDose Technologies Inc., such as the device described in United States Patent Publication 2005/0183724. Preferred devices would be those which not only disperse the powders uniformly with an active component of the device (e.g., compressed air, impeller), but also standardize the breathing profile so as to not create reverse flow rate dependence (i.e., increases in lung deposition with decreases in PIF), that is common with active DPIs.

### Capsules

Additional embodiments and features are set forth in part in the description that follows, and in part will become apparent to those skilled in the art upon examination of the specification or may be learned by the practice of the invention.

This invention is further illustrated by the following examples which should not be construed as limiting.

### Key to Abbreviations Used in the Examples

The following abbreviations are used in the Examples:

API Active Pharmaceutical Ingredient

DSPC Distearoylphosphatidylcholine

PFOB Perfluorooctyl bromide

RP-HPLC Reverse phase high performance liquid chromatography

### EXAMPLES

### Example 1

### Preparation of Spray-Blended Dry Powder Formulations of Indacaterol Maleate and Indacaterol Maleate+Mometasone Furoate

In this Example, dry powder formulations of the invention containing indacaterol maleate were prepared by a spray-blending process. This includes a formulation comprising a fixed dose combination of indacaterol maleate and mometasone furoate.

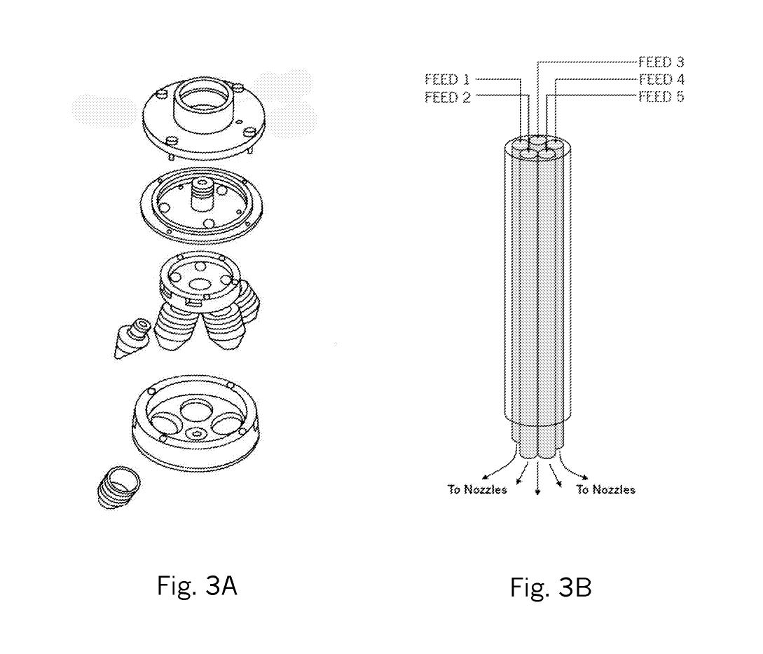

Five spray-blended formulations (see Tables 3 and 4) were prepared and spray-dried on a Niro PS-1 scale spray-drier, equipped with a multi-headed HYDRA™ atomizer. The HYDRA™ atomizer (FIG. 3) contains five twin fluid nozzles (see FIG. 3A), each of which is controlled by an independent liquid feed line as shown schematically in FIG. 3B. A common gas line was used to supply atomization gas (compressed air) to all nozzles. The nozzles are oriented to minimize interaction of the spray plumes during drying. For the five lots detailed below, only three of the five spray nozzles were utilized (Feedlines A, B, C). The composition of the feedstocks spray-dried, and the liquid flow rates are detailed in Table 3. Also shown are the compositions for indacaterol maleate (IM) and mometasone furoate (MF) in the bulk powder for the spray-blend.

For Lot I, Feedline A contained 6% w/w IM (on a free base basis). The remainder of Feedline A was comprised of a 2:1 mol:mol ratio of DSPC:CaCl₂. The feedstock for Feedlines B and C was 100% of the 2:1 mol:mol ratio of DSPC:CaCl₂. The total solids loading was 30 mg/mL for all of the spray-blended lots, and a 10:1 w/w PFOB:excipient (DSPC, CaCl₂, trehalose, sodium maleate) was utilized. The final IM content in the spray-blended formulation was 1.44%. Hence, the drug loading in the Feedline A was increased by more than 4-fold.

In Lot II, the concentration of indacaterol in Feedline A was 18% w/w and the concentration in the spray-blended bulk powder was just 4.1% w/w, as the remaining spray dried particles from Feedlines B and C contained no API. If one assumes that indacaterol maleate has a water solubility of 0.2 mg/ml, then the spray-blending process utilized in Lot II decreases the dissolved indacaterol in the feedstock from about 8.0% to 1.6% (equation 1).

Lot III was formulated like Lot II except 5% w/w trehalose was added to Feedline A. Trehalose is an excipient utilized to stabilize the amorphous IM which might form during the process.

Lot IV is formulated like Lot II except 20 mM sodium maleate (pH 5.5) was added to Feedline A. Sodium maleate was added to decrease the indacaterol solubility in water to about, 0.01 mg/ml (common ion effect). In this case, spray-blending reduced the dissolved indacaterol in the feedstock from about, 8.0% to 0.1%. Assuming that all of the % dissolved was converted to amorphous solid in the spray-dried powders, the fractions of amorphous drug introduced during the spray-drying process with spray-blending and the common ion effect are likely comparable or less than the amount introduced during standard micronization processes with an associated deamorphization step.

Lot V contains a fixed dose combination of IM and MF. The MF was formulated in Feedlines B and C.

TABLE 3
Dry powder formulations comprising indacaterol maleate (IM) or fixed
dose combinations of indacaterol maleate and mometasone furoate (MF)

| | | Feedline | Feedline | Feedline | Bulk | Bulk |
|---|---|---|---|---|---|---|
| | Feedline | B + C, | A | B + C | Powder | Powder |
| Lot | A, IM | MF | Flow rate | Flow rate | IM | MF |
| # | (% w/w) | (% w/w) | (g/min) | (g/min) | (% w/w) | (% w/w) |
| I | 6.0% | 0.0% | 24.44 | 65.0 | 1.44% | 0.00% |
| II | 18.0% | 0.0% | 18.87 | 64.4 | 4.08% | 0.00% |
| III¹ | 18.0% | 0.0% | 18.96 | 64.7 | 4.08% | 0.00% |
| IV² | 18.0% | 0.0% | 19.18 | 65.3 | 4.09% | 0.00% |
| V | 18.0% | 6.0% | 19.26 | 64.0 | 4.16% | 4.61% |

All drug contents are expressed on a free-base basis; Solids content = 30 mg/mL; 10/1 w/w PFOB/excipient ratio
¹Feedline A includes 5% w/w trehalose
²Feedline A includes 20 mM sodium maleate (pH 5.5)

The compositions of the spray-blended formulations are detailed in Table 4. Note that the formulations are made up of primarily the 2:1 mol:mol ratio of DSPC:CaCl₂ (>90% w/w). The phospholipid serves as the hydrophobic excipient, controlling the composition of the surface and morphology of the particles. It also serves as a bulking agent in the formulation.

TABLE 4
Composition of dry powder formulations of indacaterol
maleate and a fixed dose combination of indacaterol
maleate and mometasone furoate

| | Nominal content (% w/w) | | | | |
|---|---|---|---|---|---|
| Component | Lot I | Lot II | Lot III | Lot IV | Lot V |
| indacaterol maleate ¹ | 1.44 | 4.08 | 4.08 | 4.09 | 4.16 |
| Mometasone furoate ² | — | — | — | — | 4.61 |
| Trehalose | — | — | 1.36 | — | — |
| 20 mM sodium | — | — | — | 2.03 | — |
| maleate (pH 5.5) | | | | | |
| 2:1 mol:mol | Balance | Balance | Balance | Balance | Balance |
| DSPC/CaCl₂ | | | | | |

¹ Content expressed as the % w/w of the free base
² Content expressed as the % w/w of the free base

### Example 2

### Preparation of Spray-Blended Dry Powder Formulations of Indacaterol Maleate and Indacaterol Maleate+Mometasone Furoate from an Emulsion-Based Feedstock

In this Example, more detail is provided on the preparation of the feedstocks used in Example 1. The dry powder formulations of the invention containing indacaterol maleate were prepared and dry powder formulations of the invention containing indacaterol maleate and mometasone furoate were prepared from an emulsion-based feedstock that was prepared in accordance with the method described in United States patent specification U.S. Pat. No. 6,565,885. In this process, crystalline micronized indacaterol maleate is dispersed in the continuous phase of an oil-in-water emulsion. The process resulted in crystalline indacaterol particles coated with a porous layer of hydrophobic excipient. The morphology of the particles was confirmed by scanning electron microscopy (data not shown).

Accordingly, distearoylphosphatidylcholine (DSPC) and CaCl₂ are dispersed and dissolved, respectively in heated water (˜70° C.) with an ULTRA TURRAX™ T-25™ high shear mixer to form multi-lamellar liposomes. The oil phase, was perfluorooctyl bromide, PFOB (Atofina, Paris, France). PFOB was added drop-wise to the DSPC dispersion while mixing to create a coarse (micron-sized) oil-in-water emulsion. The emulsion droplets are stabilized by a monolayer of DSPC. The coarse emulsion was then homogenized under high pressure with an AVESTIN C-50® homogeniser, for three discrete passes, at pressure settings of 10, 10, and 20 kpsig. This produces fine (sub-micron) emulsion droplets. The median diameter of the emulsion droplets is typically in the range from 0.1 μm to 1.0, more typically from 0.3 μm to 0.6 μm.

An indacaterol maleate annex suspension was also prepared with the high-shear mixer. DSPC was incorporated in the dispersion as a wetting agent to facilitate suspension of indacaterol in water. The DSPC dispersion was prepared by adding DSPC to heated water (˜70° C.) and then mixing using a high-shear mixer. The DSPC dispersion was then chilled to 2-8° C., prior to addition of IM. For Lot IV, sodium maleate buffering solution was prepared by adding a predetermined amount of maleic acid and NaOH to achieve a solution with pH 5.5, which was then chilled to 2-8° C. The additional excipients for Lots III and IV were added to the DSPC dispersion prior to indacaterol addition. Indacaterol was then added to the chilled DSPC-annex dispersion using a high-shear mixer. All annex API suspensions were prepared under cold processing conditions to maintain the drug at 2-8° C. (to further minimize dissolution of the IM).

For Lot V, a mometasone furoate (MF) annex suspension was prepared using a high-shear mixer (ULTRA TURRAX™ T-25™) to disperse micronized MF in water. The feedstocks were prepared by adding the respective annexes to a fine emulsion that was maintained at 2-8° C. The resulting feedstocks wee maintained at 2-8° C. in open stainless steel vessels and mixed with an overhead LIGHTNIN® laboratory mixer. The vehicle feedstock used for Feedlines B and C was prepared by diluting the fine emulsion to a target solids concentration of 5% w/v, or 50 mg/ml.

The atomizer configuration used for this protocol allowed for three independent feedstock streams to be fed into the spray dryer. The three feedstock streams were divided as follows:
One feedstock stream (Feedline A) was used to spray dry an indacaterol-containing feedstock.
The two remaining streams (Feedlines B & C) shared the same feedstock (a Y-fitting was used to split the flow equally) which was either a vehicle (Lots I, II, III, and IV) or a mometasone-containing feedstock (Lot V).

To maintain the ratio of the feedstocks at a fixed value, a multi-headed peristaltic pump driven by a single shaft was used. A single flow meter was used to monitor the total flow rate of Feedlines B+C. Because only a single flow meter was available, the flow rate of Feedline A was determined by gravimetrically determining the mass of feedstock A delivered over a fixed time period. The spray-drying conditions and target Feedline ratios were selected following an assessment of the equipment capabilities (atomizer and multi-head feedstock pump). The target ratio of volumetric feed rates for the compositions shown in Table 3 was approximately 3:1 (Feedline B+C):(Feedline A). The target spray drying conditions are shown in Table 5.

TABLE 5
Target spray-drying conditions for preparing dry powder formulations
of indacaterol maleate using a NIRO PSD-1 scale spray-drier

| Process Parameters | Value |
|---|---|
| Inlet temperature/° C. | 140 |
| Outlet temperature/° C. | 75 |
| Collector temperature/° C. | 75 |
| Atomizer gas flow rate/L/Min | 70 |
| Total gas flow rate/scfm | 200 |

### Example 3

### Measurement of the Physical Properties of Spray-Blended Dry Powder Formulations of Indacaterol Maleate and Indacaterol Maleate+Mometasone Furoate

In this Example certain physicochemical properties of the spray-blended dry powder formulations in Example 1 were measured, namely primary particle size, tapped density and water content.

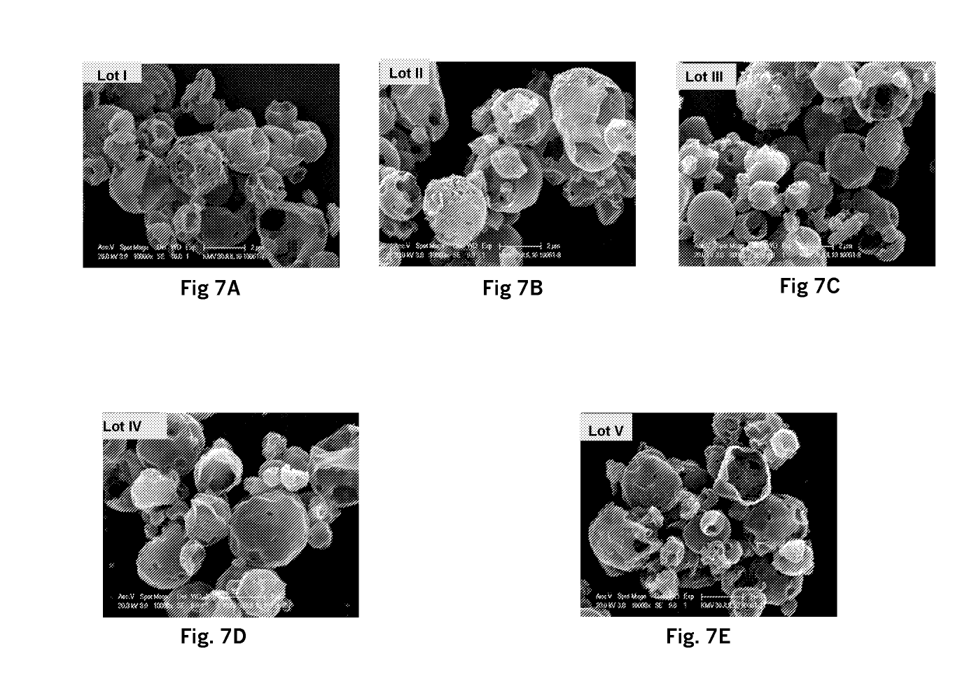

FIGS. 7A-7E are photomicrographs of spray-blended powders of embodiments of the present invention, the powders comprising indacaterol. The powders were formulated according to Table 3, and FIGS. 7A-E correspond in order to the Lot I through Lot V. The powders exhibit the hollow, porous morphology characteristic of the emulsion-based spray drying process. There is no evidence of different types of particles in the spray-blended formulations of FIGS. 7A-E.

Table 6 presents the physical properties measured for those formulations.

TABLE 6
Physical properties of spray-blended dry powder formulations of
indacaterol maleate and indacaterol maleate + mometasone furoate

| | x50 | | Tapped Density | Water content |
|---|---|---|---|---|
| Lot # | (μm) | GSD | (g/cm³) | (% w/w) |
| I | 2.7 | 1.8 | 0.04 | 2.0 |
| II | 2.7 | 1.9 | 0.04 | 1.8 |
| III¹ | 2.7 | 1.9 | 0.08 | 2.0 |
| IV² | 2.6 | 1.9 | 0.05 | 1.7 |
| V | 2.6 | 1.9 | 0.06 | 2.0 |

Primary particle size distributions of the spray-dried powders were determined via laser diffraction (Sympatec GmbH, Clausthal-Zellerfeld, Germany). The SYMPATEC HELOS™ particle size analyser was equipped with an ASPIROS™ micro dose feeder and a RODOS™ dry powder dispersing unit. A sample mass of approximately 10 mg was introduced into the ASPIROS. A triggering optical concentration (Cₒₚₜ) of approximately 1%, and a driving pressure of 4 bar were utilized. Data were collected over a measurement duration of 10 seconds. Particle size distributions were calculated by the instrument software using the Fraunhofer model. Prior to measurement of sampleₛ, the system suitability was assessed by measurement of the primary particle size distribution of a silicon carbide reference standard supplied by Sympatec GmbH. Data are presented in terms of the median diameter (x₅₀), and the GSD (x_{84.13}/x₅₀). The GSD or geometric standard deviation is a measure of the polydispersity of a log-normal particle size distribution.

Tapped densities were determined by measuring the mass of powder required to fill a cylindrical cavity (a uniaxial compaction (UC cell)) of known volume using a microspatula. The sample holder was gently tapped on the countertop. More powder was added to the cell as the sample volume decreased. The tapping and addition of powder steps were repeated until the cavity was filled and the powder bed no longer consolidated with further tapping. The reported results represent the mean of five replicates.

Water or moisture content in a powder refers to the quantity of water contained in a substance on a % w/w basis. The water content of each of the spray-blended dry powder formulations of Example 1 were determined by Karl Fischer titrimetry.

The results show the primary particle size distributions were remarkably consistent between the powders despite the fact that the compositions of the powders were very different. This demonstrates that the primary particle size distribution is controlled primarily by the spray drying conditions, and not by the differences in composition.

The physical properties of the spray-blended powders from Lots II and IV were remarkably consistent despite the dry powder of Lot II being a mono indacaterol maleate formulation and the dry powder of Lot IV being a fixed dose combination formulation of indacaterol maleate and mometasone furoate. Small differences in tapped densities noted for lot III are likely the result of the addition of trehalose to the formulation. Trehalose interacts with the lipid, thereby altering the surface properties slightly and leading to higher density particles. Mono-formulations and fixed dose combinations comprising phospholipid and added trehalose would also be expected to exhibit equivalent physical properties with comparable tapped densities. This has been demonstrated for non-spray-blended formulations.

The results show the goal of achieving spray-dried particles with physical properties independent of the composition of actives and excipients in the particles was achieved for the spray-blended dry powder formulations of Example 1.

### Example 4

### Content Uniformity of a Spray-Blended Dry Powder Containing Indacaterol Maleate

In this Example the content uniformity of the spray-blended dry powder prepared in Lot II of Example 1 was measured. Lot II contains crystalline indacaterol maleate as the sole active ingredient.

Bulk powder comprising spray-blended indacaterol from Lot II was filled into unit dose foil-foil blisters at a fill mass of 1.5 mg using a drum-based filler described in United States patent specification US 2004/0060265. The content uniformity of the filled blisters was assessed by a drug specific reversed phase high performance liquid chromatography (RP-HPLC) method. Drug content and degradation products were determined with a Waters Alliance 2695/2795 HPLC system with a Waters 2487 dual wavelength detector, Water PDA 996 detector, and Waters Empower Build 1154 software. For content and related substance analysis, a YMC Pack ODS AQ column was used; for enantiomer, a Daicel Chiral OJ-RH column was used. Two replicates for each sample were analysed. The content results are presented in Table 7 below.

TABLE 7
Content uniformity of filled blisters containing indacaterol maleate

| Lot | | Indacaterol content | Indacaterol content (based |
|---|---|---|---|
| no. | Replicate | (% w/w) | on declared content) (%) |
| II | 1 | 4.21 | 103.11 |
| | 2 | 4.12 | 101.04 |
| | 3 | 4.21 | 103.21 |
| | 4 | 4.26 | 104.47 |
| | 5 | 4.21 | 103.08 |
| | 6 | 4.16 | 101.86 |
| | 7 | 4.27 | 104.72 |
| | 8 | 4.28 | 104.89 |
| | 9 | 4.33 | 106.20 |
| | 10 | 4.26 | 104.42 |
| | | Mean | 103.70 |
| | | % RSD | 1.48 |

One replicate per 1.5 mg blister
Declared indacaterol (nominal) content = 4.08% w/w

The relative standard deviation (RSD) of the indacaterol content measurements for ten replicates is just 1.5%. The content uniformity of the filled blisters that was achieved confirmed the effectiveness of the spray-blending process of the invention in achieving a uniform blend of indacaterol particles and vehicle particles in a single step drying/blending process. The data also demonstrate the ability to hit the target indacaterol drug loading in the spray blend.

### Example 5

### Content Uniformity of a Spray-Blended Dry Powder Containing Indacaterol Maleate and Mometasone Furoate

In this Example the content uniformity of the spray-blended dry powder prepared in Lot V of Example 1 was measured. It contains indacaterol maleate and mometasone furoate as active ingredients in separate spray-blended particles.

The spray-blended dry powder from Lot V that comprise particles that contain indacaterol maleate and mometasone furoate was filled into foil-foil blisters at a fill mass of 1.5 mg. The content uniformity of the filled blisters was assessed by reverse phase high performance liquid chromatography (RP-HPLC) as described in Example 4. The results are presented in Table 8 below.

TABLE 8
Content uniformity of filled blisters containing indacaterol maleate

| | | | Indacaterol | | Mometasone |
|---|---|---|---|---|---|
| | | | content | | content |
| | | | (based on | | (based on |
| | | Indacaterol | declared | Mometasone | declared |
| Lot | | content | content) | content | content) |
| no. | Replicate | (% w/w) | (% w/w) | (%) | (%) |
| V | 1 | 4.21 | 103.11 | 4.70 | 101.88 |
| | 2 | 4.12 | 101.04 | 4.53 | 98.23 |
| | 3 | 4.21 | 103.21 | 4.60 | 99.70 |
| | 4 | 4.26 | 104.47 | 4.71 | 102.01 |
| | 5 | 4.21 | 103.08 | 4.51 | 97.86 |
| | 6 | 4.16 | 101.86 | 4.59 | 99.52 |
| | 7 | 4.27 | 104.72 | 4.59 | 99.46 |
| | 8 | 4.28 | 104.89 | 4.48 | 97.12 |
| | 9 | 4.33 | 106.20 | 4.64 | 100.56 |
| | 10 | 4.26 | 104.42 | 4.60 | 99.77 |
| | | Mean | 103.70 | Mean | 99.61 |
| | | % RSD | 1.48 | % RSD | 1.61 |

One replicate per 1.5 mg blister
Declared indacaterol (nominal) content = 4.16% w/w
Declared mometasone (nominal) content = 4.61% w/w

The uniformity of the measured content values was excellent for both active ingredients, as evidenced in each case by the RSD values for 10 replicates being 1.44% and 1.61% for indacaterol and mometasone, respectively. This confirms the effectiveness of the spray-blending process of the invention in achieving a uniform blend in a single step drying/blending process.

### Example 6

### Comparison of Emitted Powder Mass and Emitted Doses via RP-HPLC for Spray-Blended Dry Powder Formulation of Indacaterol Maleate

In this Example the aerosol performance of the spray-blended dry powder prepared in Lot II of Example 1 was measured. More specifically the Emitted Powder Mass and the Emitted Dose of the powder delivered by proprietary unit dose, passive, blister-based dry powder inhaler were determined and compared. The powder contained indacaterol maleate as the sole active ingredient.

The dry powder inhaler was the inhaler described in International Patent Application WO 08/51621. The fill mass in the blister was 1.5 mg. Aerosol performance was assessed at a pressure drop of 4 kPa, corresponding to a flow rate of 35 L/min.

The Emitted Powder Mass (EPM) and Emitted Dose (ED) of the powder were measured and the results are presented in Table 9 below.

TABLE 9
Emitted powder mass and emitted dose of the spray-blended dry powder
of Lot II containing indacaterol maleate as active ingredient

| | Gravimetric | | | Drug Specific - | |
|---|---|---|---|---|---|
| | Emitted | | Emitted | Indacaterol | |
| | Powder | Drug | Powder | Drug | Emitted |
| Replicate | Mass (mg) | (μg) | Mass (%) | (μg) | Dose (%) |
| 1 | 1.14 | 47 | 76 | 48 | 78 |
| 2 | 1.24 | 51 | 83 | 51 | 84 |
| 3 | 1.20 | 49 | 80 | 51 | 83 |
| 4 | 1.25 | 51 | 83 | 50 | 82 |
| 5 | 1.19 | 49 | 79 | 51 | 84 |
| 6 | 1.34 | 55 | 89 | 53 | 87 |
| 7 | 1.36 | 55 | 91 | 55 | 90 |
| 8 | 1.27 | 52 | 85 | 54 | 88 |
| 9 | 1.16 | 47 | 77 | 48 | 79 |
| 10 | 1.25 | 51 | 83 | 53 | 87 |
| Mean | 1.24 | 51 | 83 | 51 | 84 |
| SD | 0.07 | 3 | 5 | 2 | 4 |
| % RSD | 6 | 6 | 6 | 5 | 5 |

The gravimetric EPM determinations provide a measure of the total mass of powder (spray-dried particles containing indacaterol and spray-dried vehicle particles) that is emitted from the device and captured on a filter. Data are expressed as a percentage of the nominal fill mass.

The ED is determined by the drug specific HPLC method described in Example 4. The ED provides a measure of the mass of indacaterol exiting the inhaler, expressed as a percentage of the declared (nominal) content of indacaterol in the blister.

The results show that the EPM and ED measurements are essentially equivalent. This suggests that the drug-containing particles and the excipient/vehicle particles were well mixed during the spray-blending process. Moreover, the results demonstrate that no segregation of the two types of particles occurs during the drum filling process.

### Example 7

### Comparison of Emitted Powder Mass and Emitted Doses via RP-HPLC for Spray-Blended Dry Powder Formulation of Indacaterol Maleate and Mometasone Furoate

In this Example the aerosol performance of the spray-blended dry powder prepared in Lot V of Example 1 was measured. More specifically the Emitted Powder Mass and the Emitted Dose of the powder delivered by proprietary unit dose, passive, blister-based dry powder inhaler were determined and compared. The powder contained indacaterol maleate and mometasone furoate as active ingredients.

The dry powder inhaler was the inhaler described in International Patent Application WO 08/51621. The fill mass in the blister was 1.5 mg. Aerosol performance was assessed at a pressure drop of 4 kPa, corresponding to a flow rate of 35 L/min.

The Emitted Powder Mass (EPM) and Emitted Dose (ED) of the powder were measured and the results are presented in Table 10 below.

TABLE 10
Emitted Powder Mass and Emitted Dose of the spray-
blended dry powder of Lot V containing indacaterol
maleate and mometasone furoate as active ingredients

| | Gravimetric | | Drug Specific - | | Drug Specific - | |
|---|---|---|---|---|---|---|
| | Powder | % | Indacaterol | | Mometasone | |
| | Mass | Emitted | Drug | % Emitted | Drug | % Emitted |
| Replicate | (mg) | Mass | (μg) | Dose | (μg) | Dose |
| 1 | 1.05 | 71 | 49 | 80 | 52 | 76 |
| 2 | 1.21 | 82 | 55 | 89 | 58 | 85 |
| 3 | 1.09 | 74 | 48 | 78 | 51 | 75 |
| 4 | 1.24 | 84 | 54 | 88 | 57 | 84 |
| 5 | 1.26 | 85 | 55 | 90 | 58 | 85 |
| 6 | 1.25 | 84 | 54 | 88 | 59 | 86 |
| 7 | 1.14 | 77 | 51 | 83 | 53 | 78 |
| 8 | 1.07 | 72 | 46 | 75 | 49 | 72 |
| 9 | 1.28 | 86 | 56 | 91 | 58 | 85 |
| 10 | 1.30 | 88 | 51 | 84 | 58 | 85 |
| Mean | 1.19 | 80 | 52 | 84 | 55 | 81 |
| SD | 0.09 | 6 | 3 | 5 | 4 | 5 |
| % RSD | 8 | 8 | 6 | 6 | 6 | 6 |

The results show the gravimetric EPM and drug-specific ED determinations correspond closely. This suggests that the content enriched indacaterol particles were well mixed with the particles containing mometasone during the spray-blending process. Moreover, the results demonstrate that no segregation of the two types of particles occurs during the drum filling process.

### Example 8

### Aerodynamic Particle Size Distribution of a Spray-Blended Dry Powder Formulation of Indacaterol Maleate and Mometasone Furoate

In this Example the aerosol performance of the spray-blended dry powder prepared in Lot V of Example 1 and delivered by a proprietary unit dose, passive, blister-based dry powder inhaler was determined by measuring its Aerodynamic Particle Size Distribution (APSD).

The dry powder inhaler was the inhaler described in International Patent Application WO 08/51621. The experimentally determined nominal doses were 70±1 mcg for the two drugs. The fill mass in the blister was 1.5 mg. The flow rate was 35 L/min, which corresponds to a pressure drop of 4 kPa.

APSD were measured with a NEXT GENERATION IMPACTOR™. Stage-by-stage powder masses of indacaterol and mometasone were assessed for the powder. Statistics (mean, standard deviation) are based upon five replicate measurements.

The results of the APSD measurements are presented in Table 11 below.

TABLE 11
Aerodynamic particle size distribution of the spray-
blended dry powder of Lot V containing indacaterol
maleate and mometasone furoate as active ingredients

| | Indacaterol per stage/ | | Mometasone per stage/ | | |
|---|---|---|---|---|---|
| | blister | | blister | | |
| NGI | Mean | Std Dev | Mean | Std Dev | |
| Stage | (μg) | (μg) | (μg) | (μg) | % Difference |
| 0 | 3.46 | 0.71 | 3.91 | 0.70 | |
| 1 | 2.33 | 1.43 | 2.50 | 1.38 | |
| 2 | 2.94 | 0.96 | 3.13 | 0.94 | |
| 3 | 16.43 | 1.78 | 16.41 | 1.85 | |
| 4 | 24.02 | 1.07 | 22.18 | 1.63 | |
| 5 | 6.69 | 0.38 | 7.03 | 0.45 | |
| 6 | 0.63 | 0.12 | 0.80 | 0.13 | |
| 7 | 0.05 | 0.03 | 0.06 | 0.03 | |
| 8 | 0 | 0.00 | 0.00 | 0.00 | |
| LPD₀₋₂ | 8.7 | | 9.5 | | 8.5 |
| FPD_{3-F} | 47.8 | | 46.5 | | 2.8 |
| VFPD_{4-F} | 31.4 | | 30.1 | | 4.2 |

The aerodynamic particle size distribution was divided into various stage groupings. “LPD₀₋₂” means the large particle fraction present on stages 0-2 (mass basis). “FPD_{3-F}” refers to the fine particle dose on stages 3 to filter, and “VFPD_{4-F}” represents the very fine particle dose on stages 4 to filter. The mass of indacaterol and mometasone on the various stages in the NGI were determined by a drug specific HPLC method.

The results show the APSD for indacaterol and mometasone correspond very closely on a stage by stage basis. The small differences in LPD, FPD, and VFPD for the two active ingredients indicate the powder has excellent blend uniformity. Moreover, the excellent agreement in APSD for the spray-blended powders indicates that the differences in particle properties are negligible between the two powders. This was of course by design in that the particles were designed to have a core-shell structure where the surface composition and morphology are controlled by the hydrophobic excipient (DSPC), with the differing APIs present in the core of the particles.

The total deposition of the two active ingredients on stage 3 to filter represents 66%-68% of the measured nominal dose. Based on previous gamma scintigraphy studies in healthy volunteers, it would be anticipated that lung delivery in-vivo will be ˜50-60% of the nominal dose with an interpatient variability of just 10-20%.

### Example 9

### Chemical Stability of a Spray-Blended Dry Powder Containing Indacaterol Maleate

In this Example the chemical stability of all of the spray-blended dry powders prepared in Example 1 and six other spray-blended dry powders containing indacaterol maleate was assessed using RP-HPLC, as described in Example 4.

The bulk powders were assessed under accelerated conditions (T=60° C.) over a period of seven days. The results are presented in Table 12 below. They show the increases in degradation products, i.e., enantiomer content and total degradation products.

TABLE 12
Chemical stability of spray-blended formulations comprising
indacaterol maleate (T = 60° C., 7 days)

| | | | | Total |
|---|---|---|---|---|
| | Composition | *Estimated | Enantiomer | Impurities |
| Lot # | (% w/w) | % Dissolved | (% w/w) | (% w/w) |
| I | 1.44% indacaterol | 4.9 | 1.82 | 2.70 |
| | maleate* | | | |
| II | 4.08% indacaterol | 1.6 | 0.60 | 1.2 |
| | maleate* | | | |
| III | 4.08% indacaterol | 1.6 | 0.48 | 1.1 |
| | maleate, 5% trehalose* | | | |
| IV | 4.16% indacaterol | 1.6 | 0.38 | 0.8 |
| | maleate, 4.61% | | | |
| | mometasone furoate* | | | |
| V | 4.09% indacaterol | 0.08 | 0.42 | 0.6 |
| | maleate, | | | |
| | 20 mM maleate, pH 5.5* | | | |
| VI | 2% indacaterol maleate | 14.8 | 7.7 | 4.7 |
| VII | 6% indacaterol maleate, | 5.3 | 1.8 | 1.4 |
| | 5% solids | | | |
| VIII | 2% indacaterol maleate, | 0.7 | 1.41 | 1.9 |
| | 20 mM maleate, pH 5.5 | | | |
| IX | 6% indacaterol maleate, | 0.3 | 0.59 | 0.8 |
| | 20 mM maleate, pH 5.5 | | | |
| X | 3% indacaterol maleate, | 10.0 | 3.0 | 2.3 |
| | 5% solids | | | |
| XI | 6% indacaterol maleate, | 8.9 | 2.9 | 2.3 |
| | 3% solids | | | |

*Spray-blended powders; Lots VI to XI were prepared according to the process described in Example 2, although the powders were spray-dried from a single feedstock.

The chemical stability is quantitated in terms of the percentage of conversion to the S-enantiomer of indacaterol and in total impurities observed via HPLC.

Estimated % Dissolved was calculated based on the feedstock compositions according to equation 1. The solubility of indacaterol in water is 0.2 mg/ml, and 0.01 mg/ml in 20 mM maleate buffer. The decreased solubility in maleate buffer is the result of the shift in the equilibrium towards precipitation of indacaterol maleate which occurs as a result of the addition of a common ion. The impact of spray-blending on the dissolved fraction is clearly evident by comparing Lots I and VI. In the spray-blended Lot I, the total drug loading is 1.4%, yet the dissolved fraction is just 4.9%. In contrast, for the single feedstock Lot VI, the drug content is actually higher (2%), yet the dissolved fraction is nearly 15%. The higher dissolved fraction results in significantly higher levels of enantiomer (7.7% vs. 1.8%), and higher levels of total impurities (4.7% vs. 2.7%). Lot V utilizing both the common ion effect and spray-blending shows the lowest dissolved fraction and best chemical stability of the lots tested.

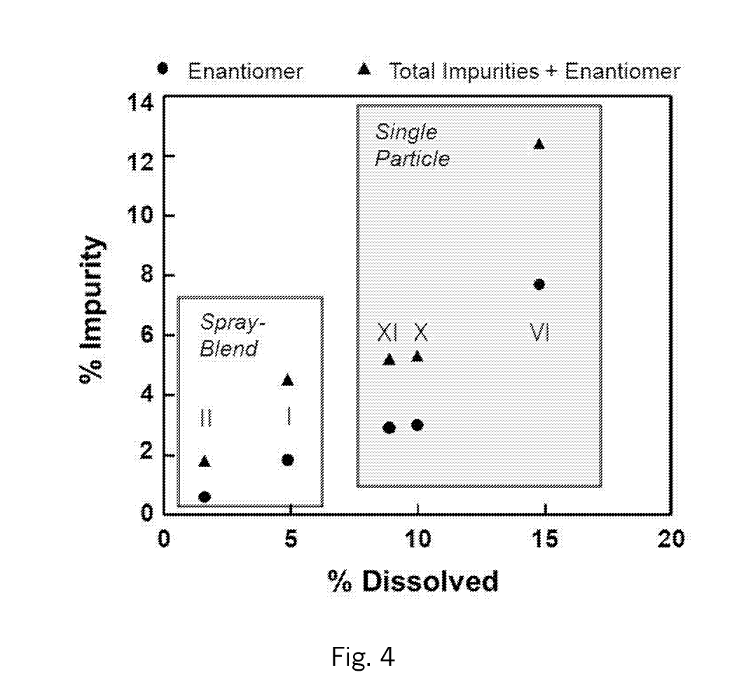

The results shown in Table 12 show a strong correlation between the dissolved fraction of indacaterol in the spray-drying feedstock, and the resulting measures of indacaterol chemical stability on storage. The results are plotted in FIG. 4.

FIG. 4 shows the % dissolved indacaterol for Lots I-XI plotted against enantiomer and eneatiomer plus total impurities. Significant improvements in chemical stability are noted with decreases in the dissolved content of indacaterol. The addition of a common ion (maleate) shifts the equilibrium towards the salt, significantly decreasing its solubility (common ion effect). This also is a means to decrease the dissolved fraction of indacaterol. When the dissolved fraction is low, the addition of a glass-forming agent (e.g., trehalose) provides little added benefit, as the amorphous content in the powder is low.

Together the results presented in Table 12 and FIG. 4 show a significant correlation between the fraction of indacaterol which is dissolved in the feedstock to be spray-dried and the resulting chemical stability on storage. It is presumed that the reduced chemical stability occurs as a result of an increase in the fraction of amorphous indacaterol in the powder. Owing to the fast dry times associated with spray-drying (millisecond timescale), it is presumed that any indacaterol which dissolves in the feedstock will be converted into amorphous material in the spray-dried drug product. Unfortunately, it is not possible to quantitate the amorphous content in the spray-dried powders, due to the low drug loadings and poor sensitivity of current analytical methods. Nonetheless, the probability that dissolved indacaterol is converted into amorphous drug in the spray-dried powders is high, and the correlation between the dissolved fraction of indacaterol and the resulting differences in stability observed on storage provides additional evidence for the link between dissolved fraction/amorphous content/chemical stability.

### Example 10

### Estimates of Lung Delivery of Spray-Dried Indacaterol Maleate Engineered Powders in the Breezhaler® Dry Powder Inhaler

Example 10 provides estimates of the anticipated mean lung deposition in-vivo from measurements of the mass of active ingredient which deposits on a filter past the idealized Alberta mouth-throat. The idealized Alberta mouth-throat model was developed based on the casts of mouth-throat anatomies obtained from imaging studies. The model was designed to provide an average deposition for the mouth-throat. The “lung dose” represents the mass of active ingredient which is not deposited in the mouth-throat.

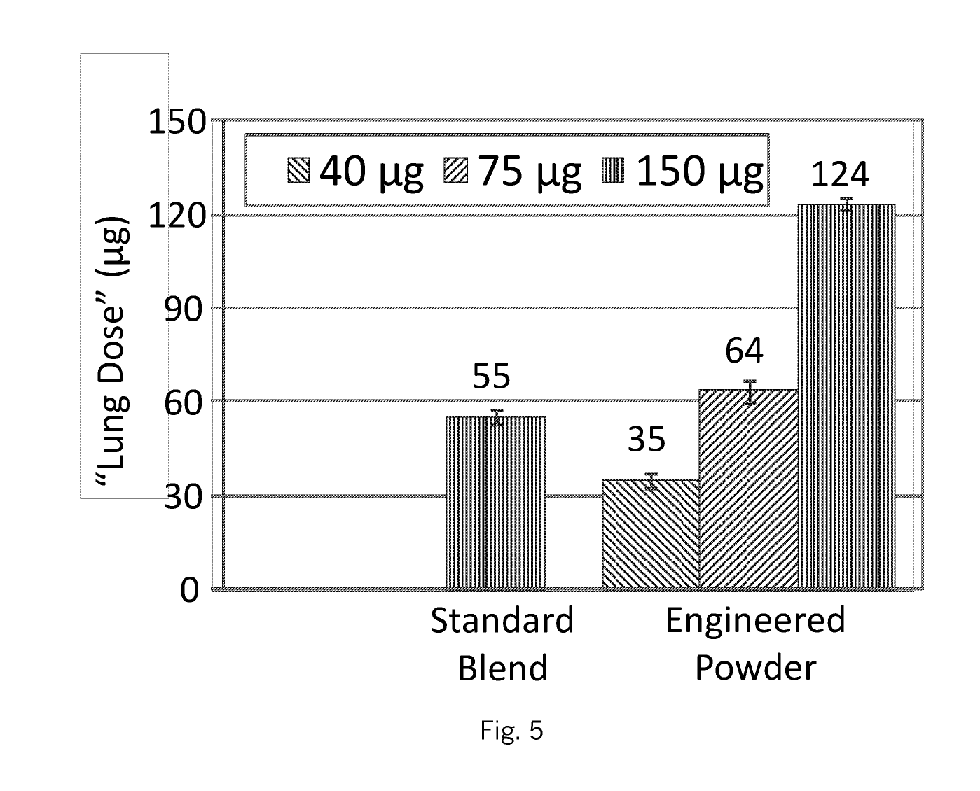

The in-vitro “lung dose” for a spray-dried formulation of IM (Lot VIII) is presented in FIG. 5. The engineered powder is delivered with the Breezhaler® dry powder inhaler. The Breezhaler®is a portable, capsule-based dry powder inhaler with a low device resistance. The results are compared with the results from the marketed IM product (OnBrez® inhalation powder, Novartis, Basel, Switzerland) which is formulated using standard blend technologies, and which utilizes the same dry powder inhaler. The in-vitro lung dose for the engineered powder is about twice that of the standard blend. The 37% lung deposition predicted for the commercial OnBrez drug product agrees well with previous pharmacokinetic results for this drug product in COPD patients. Hence, these results suggest that lung deposition for the engineered powders should be about 70% of the nominal dose. Moreover, the engineered powders show a linear dose response.

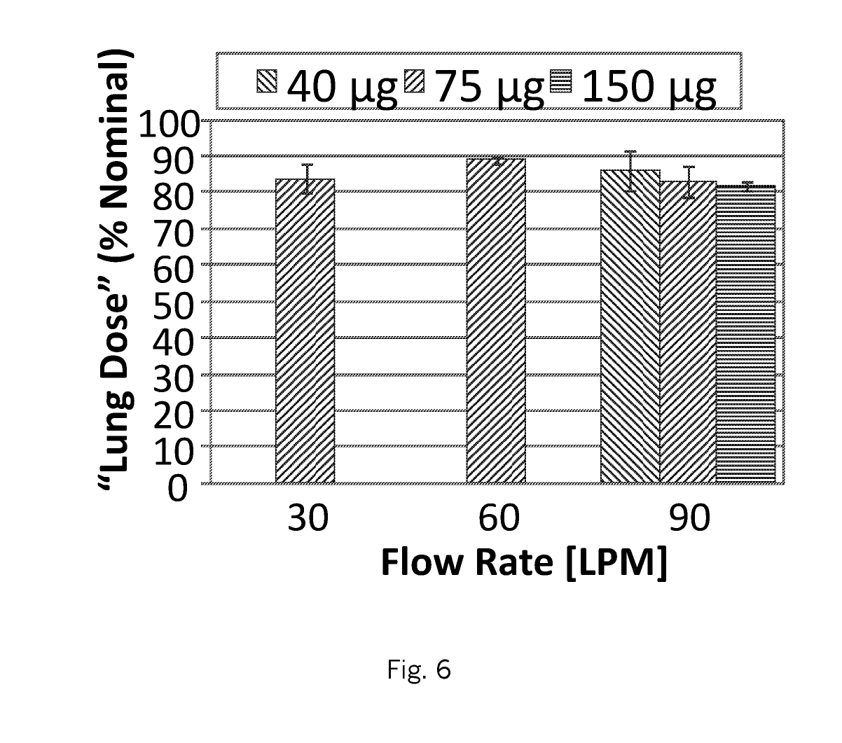

The engineered powder formulation (Lot VIII) also shows a minimal dependence on flow rate. FIG. 6 shows a plot of the in-vitro lung dose as a function of flow rate through the Breezhaler® dry powder inhaler. The flow rate is varied from 30 L/min to 60 L/min to 90 L/min. The Breezhaler® inhaler is a low resistance device, and most patients can achieve flow rates in excess of 90 L/min. Hence, the 30 L/min flow rate represents a very stringent test condition. The in-vitro lung dose stays above 80% for all of the flow rates tested.

### Example 11

### Aerodynamic Particle Size Distributions in Spray-Blend Formulations of Indacaterol Maleate and Mometasone Furoate Delivered from the Breezhaler®Dry Powder Inhaler

The aerodynamic particle size distribution of a fixed dose combination of IM and MF (Lot V) from the Breezhaler® dry powder inhaler was compared with results from the mono IM formulation (Lot II). Results are presented in terms of the MMAD and the mass on stage grouping from S3-F (Table 13) obtained on a Next Generation Impactor. The Breezhaler® inhaler was operated at a flow rate of 60 L/min. The fill mass was adjusted to deliver a nominal dose of about 150 μg.

The MMAD and FPF_{S3-F} are consistent for IM in the mono and combo formulations. Moreover, the delivery of IM and MF are consistent within the combination product. The overall deviation in FPF_{S3-F} for IM and MF in the combo product is less than 5% from the drug delivery obtained for the mono formulation.

TABLE 13
Emitted powder mass and emitted dose of the spray-blended dry powder
of Lot II containing indacaterol maleate as active ingredient

| | | MMAD | FPF_{S3-F} | |
|---|---|---|---|---|
| Lot # | API | (μm) | (%) | Difference |
| II | IM | 2.9 | 70.7 | — |
| V | IM | 2.8 | 73.9 | +4.5% |
| | MF | 2.7 | 71.4 | +1.0% |

### Example 12

### Compositions of Indacaterol Maleate, Mometasone Furoate, and Glycopyrronium Bromide Prepared by Spray-Blending

Six additional lots of spray-blended powders were manufactured on a Niro PS-1 scale spray-drier, equipped with a multi-headed Hydra atomizer. All of the lots were formulated using a 2:1 mol:mol ratio of distearoylphosphatidylcholine (DSPC):CaCl₂, and a 10:1 PFOB:excipient (DSPC and CaCl₂) mass ratio. The placebo feedstock comprised DSPC and CaCl₂ at a total solids concentration of 4.04% w/w and with a PFOB to water mass ratio of 0.68 w/w. The drug substance feedstock comprised indacaterol maleate (IM), glycopyrronium bromide (GB) and mometasone furoate (MF), DSPC and CaCl₂ at a total solids concentration of 4.19% w/w and with a PFOB to water mass ratio of 0.52 w/w for all lots. The placebo and drug substance feedstock compositions of the six lots are detailed in Table 14. The placebo and drug feedstocks were spray dried at a feed ratio of approximately 3:1, respectively. The two feedstocks were spray blended on a Niro PSD-1 where the placebo feedstock was pumped at a rate ranging from 63.7 to 75.1 g/min, and the drug substance was pumped at rates ranging from 22.5 to 24.9 g/min. The target compositions for the 6 spray-blended lots are listed in Table 15. The drug containing particles are enriched by more than 5-fold in drug compared to the bulk composition, thereby decreasing the dissolved fraction for poorly soluble drugs (e.g., indacaterol maleate) in the feedstock. Moreover, the addition of sodium maleate as a common ion decreases indacaterol maleate solubility from 0.2 mg/ml to 0.01 mg/ml. Overall, the % dissolved for indacaterol maleate in the six lots was ≈0.17% (calculated using equation 1).

TABLE 14
Spray-blend formulations comprising indacaterol maleate and its fixed dose combinations

| | | Placebo | | | | | | | |
|---|---|---|---|---|---|---|---|---|---|
| | | Feedstock | | | | | | | |
| | | Composition¹ | Drug Substance Feedstock Composition¹ | | | | | | |
| | | DSPC + | DSPC + | | Maleic | Citric | | | |
| Lot | Formulation | CaCl₂ | CaCl₂ | NaOH | Acid | Acid | IM² | GB³ | MF |
| ID | Description | (w/w) | (w/w) | (w/w) | (w/w) | (w/w) | (w/w) | (w/v) | (w/w) |
| 6-1 | IM/GB/MF Trombo | 100% | 69.2% | 1.2% | 3.7% | — | 10.4% | 7.5% | 8.0% |
| | (Maleate buffer, pH 3) | | | | | | | | |
| 6-2 | IM/GB/MF Trombo | 100% | 69.2% | 1.3% | 3.6% | — | 10.4% | 7.5% | 8.0% |
| | (Maleate Buffer, pH 4.5) | | | | | | | | |
| 6-3 | IM/GB/MF Trombo | 100% | 67.7% | 1.7% | 1.8% | 3.0% | 10.4% | 7.5% | 8.0% |
| | (Maleate & Citrate | | | | | | | | |
| | Buffers, pH 4.5) | | | | | | | | |
| 6-4 | IM/GB Combo | 100% | 77.2% | 1.2% | 3.7% | — | 10.4% | 7.5% | — |
| | (Maleate Buffer, pH 3) | | | | | | | | |
| 6-5 | IM/MF Combo | 100% | 76.9% | 1.2% | 3.7% | — | 10.4% | — | 8.0% |
| | (Maleate Buffer, pH 3) | | | | | | | | |
| 6-6 | IM | 100% | 84.8% | 1.2% | 3.7% | — | 10.4% | — | — |
| | (Maleate Buffer, pH 3) | | | | | | | | |

¹Feedstock components are expressed on an anhydrous basis.
²IM/base ratio on anhydrous basis is 1.296
³GB Salt/base ratio on anhydrous basis is 1.251

TABLE 15
Formulated Spray-Blended Powder Compositions

| | | Target Spray-Dried Powder Composition⁴ | | | | | |
|---|---|---|---|---|---|---|---|
| | | DSPC + | Maleic | Citric | | | |
| Lot | Formulation | CaCl2 | Acid | Acid | IM | GB | MF |
| ID | Description | (w/w) | (w/w) | (w/w) | (w/w) | (w/w) | (w/w) |
| 6-1 | IM/GB/MF Trombo | 93.58% | 0.76% | — | 2.06% | 1.54% | 2.06% |
| | (Maleate buffer, pH 3) | | | | | | |
| 6-2 | IM/GB/MF Trombo | 93.60% | 0.74% | — | 2.06% | 1.54% | 2.06% |
| | (Maleate Buffer, pH 4.5) | | | | | | |
| 6-3 | IM/GB/MF Trombo | 93.35% | 0.37% | 0.62% | 2.06% | 1.54% | 2.06% |
| | (Maleate & Citrate | | | | | | |
| | Buffers, pH 4.5) | | | | | | |
| 6-4 | IM/GB Combo | 95.64% | 0.76% | — | 2.06% | 1.54% | — |
| | (Maleate Buffer, pH 3) | | | | | | |
| 6-5 | IM/MF Combo | 95.12% | 0.76% | — | 2.06% | — | 2.06% |
| | (Maleate Buffer, pH 3) | | | | | | |
| 6-6 | IM | 97.18% | 0.76% | — | 2.06% | — | — |
| | (Maleate Buffer, pH 3) | | | | | | |

⁴Bulk Powder compositions are expressed on an anhydrous basis.

The spray-blended particle formulations are spray-dried from an emulsion-based feedstock utilizing a multi-headed Hydra atomizer. Micronized indacaterol maleate and mometasone furoate are dispersed in the continuous phase of the oil-in-water emulsion of the drug substance feedstock; whereas, glycopyrronium bromide, maleic acid, citric acid and the sodium hydroxide are dissolved in the continuous phase. The maleic acid was added to suppress the solubility of indacaterol maleate by means of the common ion effect and to buffer the formulation. Citric acid is added as a buffer to batch 6-3 to control the pH at 4.5 since maleic acid has little or no buffering capacity at the desired pH. All drug substance feedstock pHs were adjusted using sodium hydroxide. The drug substance feedstocks were co-spray-dried with a placebo feedstock using a Hydra atomizer equipped with four nozzles at a feed ratio of approximately 1:3, respectively. The target compositions of the feedstocks are described in Example 12 (Table 14). The manufacturing process results in particles comprising amorphous GB, crystalline IM and MF coated with a porous layer of hydrophobic excipients.

The placebo feedstock was prepared by dispersing distearoylphosphatidylcholine (DSPC) in heated water (˜70° C.) containing dissolved CaCl₂ with a high-shear mixer (Ultra-Turrax T-50, IKA-Werke GmbH, Staufen Germany) to form multilamellar liposomes. Perfluorooctyl bromide (PFOB) was added to the DSPC dispersion while mixing to create a coarse (micron-sized) oil-in-water emulsion. Additional water was added to the coarse emulsion to obtain the required emulsion weight to account for evaporative losses. The coarse emulsion was then homogenized (M110 Microfluidizer, Microfluidics Corp., Newton, Mass.) in two discrete passes at pressure settings of 20±3 kpsig to create a sub-micron emulsion.

The drug substance feedstocks were prepared by dispersing IM and/or MF drug substance crystals into oil-in-water emulsion comprising sodium maleate, DSPC, calcium chloride and PFOB using a high-shear mixer (Ultra-Turrax T-25, IKA-Werke GmbH, Staufen Germany). All drug substance feedstocks were maintained at 2 to 8° C. As required, GB was added and dissolved in the continuous phase of the oil-in-water emulsion. For batch 6-3, the citrate buffer was prepared and added to the oil-in-water emulsion along with maleic acid prior to addition of the IM and MF drug substances.

The oil-in-water emulsion for the drug substance feedstocks were prepared using the same procedures and equipment as described above for the placebo feedstock. The oil-in-water emulsion was then chilled to 2-8° C. For each batch except for 6-3, a sodium maleate buffering solutions were prepared by adding a predetermined amount of maleic acid and NaOH to achieve a solution at pH 3, which was then chilled to 2-8° C. For batch 6-3, the buffering solution was prepared by adding a predetermined amount of citric acid and maleic acid and NaOH to achieve a pH of 4.5, which was then chilled to 2-8° C.

The atomizer configuration used for this protocol allowed for four independent feedstock lines to be fed into the spray dryer. The four feedstock streams were divided as follows:
Drug substance feedstocks were fed into one of the atomizer streams (Low flow).
The Placebo feedstocks were fed into three remaining atomizer streams equipped with a cascading Y-fittings to split the flow (High flow).

To maintain the ratio at which the feedstocks were pumped, two independently controlled peristaltic pumps were used. Each feedstock flow rates were monitored. The target spray drying conditions are shown in Table 16.

TABLE 16
Target spray-drying conditions for spray-blended formulations
comprising indacaterol maleate, mometasone furoate and glycopyrronium
bromide on a Niro PSD-1 scale spray-drier

| Inlet | Outlet | Collector | Atomizer gas | Total Gas |
|---|---|---|---|---|
| temperature | temperature | temperature | flow rate | flow rate |
| (° C.) | (° C.) | (° C.) | (scfm) | (scfm) |
| 140 | 75 | 70 | 70 | 200 |

### Example 13

### Aerosol Performance of Spray Blended Combinations

The aerosol performance for indacaterol maleate in selected spray-blended lots are presented in Table 17. The powders were delivered with a portable, passive dry powder inhaler (T-326), at a flow rate of 60 L/min. The formulations comprise the mono IM formulation, its fixed dose combinations with MF and GB, and the triple combination of IM/GB/MF. The aerosol performance is consistent between the mono formulation and the fixed dose combinations with the variation in fine particle fraction (FPF_{S3-F}) for the fixed dose combinations relative to the mono formulation of 10% or less.

TABLE 17
Aerosol performance of indacaterol maleate
in spray-blended formulations

| | | MMAD | FPF_{S3-F} | |
|---|---|---|---|---|
| Lot # | Formulation | (μm) | (% nominal) | Difference |
| 6-6 | IM | 2.6 | 68 | — |
| 6-5 | IM/MF | 2.8 | 74 | +8.9% |
| 6-4 | IM/GB | 2.7 | 75 | +10.3% |
| 6-1 | IM/MF/GB | 2.7 | 70 | +2.9% |

### Example 14

### Chemical Stability of Spray-Blended Formulations Comprising IM, MF, and GB

The chemical stability of spray-blended formulations of IM and its fixed dose combinations with MF and GB are presented in Table 18. The data presented represents the major degradation products for each of the three drug substances as determined by RP-HPLC. Spray-blending has maintained the crystalline nature of indacaterol (% dissolved 0.17%) resulting in minimal chemical degradation on storage. The only degradation product which appears at levels significantly above the LOQ is the 529 peak for indacaterol. This is the enantiomeric form of the drug. The enantiomer has been qualified in preclinical studies to much higher levels, and this degree of degradation is not a concern.

TABLE 18
Chemical stability of spray-blended formulations of IM, MF
and GB. The values represent the degradation products measured
at 9 months following storage at 25° C. and 60% RH

| | Degradation Product (% w/w) | | | | | |
|---|---|---|---|---|---|---|
| | | | 529 | | | |
| | | | (enan- | | | |
| Formulation | 513 | 520 | tiomer) | 543 | Cmpd 1 | Cmpd E |
| Fill mass = | IM | | | GB | MF | |
| 2 mg | Related | | | Related | Related | |
| 6-6 | BLQ | BLQ | 0.30 | — | — | — |
| IM | (<0.05) | (<0.05) | | | | |
| 6-5 | BLQ | BLQ | 0.32 | — | BLQ | BLQ |
| IM/MF | (<0.05) | (<0.05) | | | (<0.05) | (<0.05) |
| 6-4 | BLQ | 0.07 | 1.16 | BLQ | — | — |
| IM/GB | (<0.05) | | | (<0.10) | | |
| 6-1 | BLQ | 0.08 | 1.04 | BLQ | BLQ | BLQ |
| IM/MF/GB | (<0.05) | | | (<0.10) | (<0.05) | (<0.05) |

### Example 15

### Impact of Compound X Dissolution in Water on Chemical Stability of Spray-Dried Drug Product

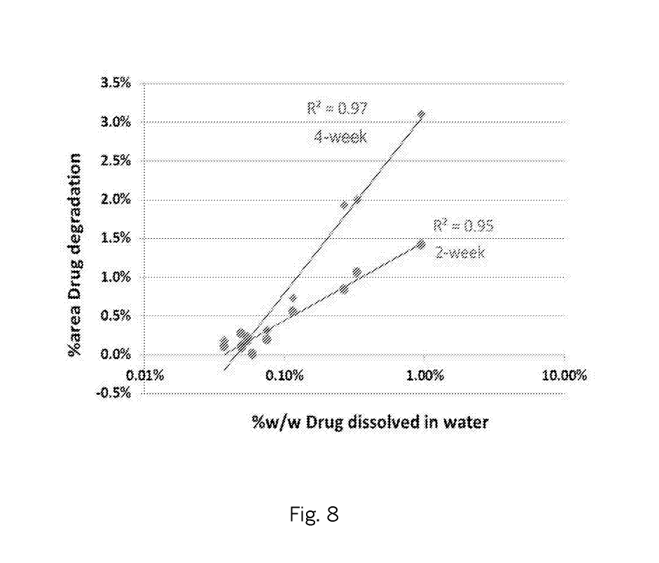

This Example illustrates that the spray-blending methods and compositions of the present invention can be applied to any suspension-based spray-drying process, where the API that has a finite solubility in the aqueous feedstock to be spray-dried. In this Example a novel prostacyclin analog (compound X) for the treatment of pulmonary arterial hypertension is spray blended. The free base form of compound X has a solubility in water of 0.01 mg/mL. As the dose of the API is pushed down into the 100 mcg range, the drug loading of the formulation must also be decreased. This results in dissolution of API in the feedstock to be spray-dried. Even small amounts of dissolved compound X (about 1% w/w) can have a significant impact on the chemical stability of the spray-dried drug product. FIG. 8 shows a plot of API degradation observed for compound X as a function of the percent dissolved over two week and four week time periods. In FIG. 8, it can be seen that small amounts of API dissolution have a large impact on chemical stability of the spray-dried drug product. Percent dissolved is varied via changes in the drug content and solids content in the feedstock (See Table 19 below). The balance of the formulation is a 2:1 molar ratio of DSPC:CaCl₂. All of the formulations were spray-dried on a custom laboratory scale spray-drier designed by Novartis scientists. The spray-dried powders were stored at 40° C./75% RH over these time periods. Significant increases in degradation are observed for % dissolved exceeding 0.1%. Hence in some embodiments a method and composition of the present invention a % dissolved active is less than about 0.1%, such as less than about 0.09%, or 0.08% or 0.07% or 0.06% or 0.05%. In some embodiments of a method and composition of the present invention, a percentage drug degradation after 4 weeks is less than about 1.5%, such as less than about 1% or 0.9% or 0.8% or 0.7% or 0.6% or 0.5% or 0.4% or 0.3% or 0.2% or 0.1%.

TABLE 19
Impact of variations in Percent Dissolved in compound X degradation

| | | | PFOB ratio | Solid conc. | | | |
|---|---|---|---|---|---|---|---|
| | Drug | Drug | in liquid | in liquid | % Drug | % Area | |
| | content | content | (water + | (water + | dissolved | degradation at | |
| Lot | (Target) | (Actual) | PFOB) | PFOB) | in water | 40° C./75% RH | |
| # | % w/w | % w/w | % v/v | % w/v | % w/w | 2 wk | 4 wk |
| 477-58-01 | 2.5% | 2.66% | 18.3% | 3.0% | 0.952% | 1.42% | 3.10% |
| 477-58-02 | 10.0% | 10.30% | 16.9% | 3.0% | 0.289% | 0.84% | 1.93% |
| 477-58-03 | 40.0% | 39.17% | 11 3% | 3.0% | 0.075% | 0.19% | 0.31% |
| 477-58-04 | 5.0% | 4.53% | 19.2% | 5.4% | 0.330% | 1.07% | 2.00% |
| 477-58-05 | 15.0% | 13.25% | 17.7% | 5.4% | 0.115% | 0.56% | 0.73% |
| 477-58-06 | 45.0% | 43.79% | 11.8% | 5.4% | 0.037% | 0.11% | 0.18% |
| 477-63-01 | 30.0% | 32.62% | 13.8% | 5.4% | 0.049% | 0.27% | 0.13% |
| 477-63-02 | 30.0% | 33.46% | 12.3% | 4.8% | 0.055% | 0.18% | 0.23% |
| 477-63-03 | 50.0% | 48.34% | 14.1% | 3.0% | 0.059% | 0.01% | −0.01% |
| 477-63-04 | 60.0% | 59.83% | 11.3% | 3.0% | 0.049% | 0.10% | 0.09% |

### Example 16

### Spray-Blending of Compound X Formulations to Maintain Chemical Stability of Spray-Dried Drug Product

In order to maintain the stability of Compound X, a spray-blending process was developed wherein particles comprising Compound X at a drug loading of 20% and 40% w/w to limit API dissolution, were mixed with PULMOSPHERE™ placebo particles containing a 2:1 molar ratio of DSPC:CaCl₂. The spray-blended formulations had an API content as low as 2.5% w/w. Table 20 provides the compositions for the spray-blended formulations tested. All of the formulations were prepared on a Niro PSD-1 scale spray-drier equipped with custom atomization and collection hardware. The Hydra atomizer was used to spray-blend up to five independent liquid feed and atomization gas streams.

TABLE 20
Stability Testing of Spray Blended Formulations

| | | | | | Powder | |
|---|---|---|---|---|---|---|
| | | | | Powder | spray | |
| | Feedstock stream | | Placebo | target | blend | Drug |
| | Drug | | stream | Drug | Drug | dissolved |
| LOT | loading | Solids | PFOB/ | loading | loading | in water |
| ID | % w/w | % w/v | DSPC | % w/w | % w/w | % w/w |
| 12118B-2-1 | 20 | 3.0 | 9.0 | 2.5 | 2.6 | 0.142 |
| 12118B-2-2 | 20 | 5.4 | 9.0 | 2.5 | 2.5 | 0.068 |
| 12118B-2-3 | 40 | 3.0 | 9.0 | 2.5 | 2.5 | 0.074 |
| 12118B-2-4 | 40 | 5.4 | 9.0 | 5.0 | 5.0 | 0.037 |
| 12118B-2-5 | 20 | 5.4 | N/A | 20.0 | 19.9 | 0.068 |
| 12118B-2-6 | 20 | 5.4 | 4.0 | 2.5 | 3.0 | 0.068 |
| 12118B-2-7 | 40 | 5.4 | 9.0 | 20.0 | 21.0 | 0.037 |

Following storage for 4 weeks at 40° C./75% RH, the total degradation was less than 0.35% for all of the spray-blends tested. For formulations where the nozzle containing Compound X was maintained at a 40% w/w concentration, the total degradation was less than 0.20%. Hence, the spray-blending process was effective in minimizing API dissolution in the feedstock, and in maintaining the chemical stability of the spray-dried drug product.

The various features and embodiments of the present invention, referred to in individual sections above apply, as appropriate, to other sections, mutatis mutandis. Consequently features specified in one section may be combined with features specified in other sections, as appropriate.

Those skilled in the art will recognize, or be able to ascertain using no more than routine experimentation, many equivalents to the specific embodiments of the invention described herein. Such equivalents are intended to be encompassed by the following claims.

1. A powder formulation for inhalation, comprising a substantially uniform blend of a first engineered powder and a second engineered powder,
said first engineered powder comprising first spray-dried particles comprising a crystalline therapeutically active ingredient dispersed in a pharmaceutically acceptable hydrophobic excipient;
said second engineered powder comprising second spray-dried particles of a pharmaceutically acceptable hydrophobic excipient, wherein the second spray-dried particles are substantially free of any therapeutically active ingredient; and
wherein a loading of the active ingredient in said first engineered powder is sufficiently high to achieve a desired target dose of the active ingredient.

2. A formulation according to claim 1, wherein the active ingredient is selected from the group consisting of bronchodilators, anti-inflammatories, antihistamines, decongestants, anti-tussive drug substances and prostacyclin analogs.

3. A formulation according to claim 2, wherein the active ingredient is an indacaterol salt, a glycopyrronium salt or mometasone salt.

4. A formulation according to claim 1, wherein the first powder contains two or more active ingredients selected from the group consisting of bronchodilators, anti-inflammatories, antihistamines, decongestants and anti-tussive drug substances.

5. A formulation according to claim 4, wherein the first powder contains an indacaterol salt and glycopyrrolate as active ingredients.

6. A formulation according to claim 4, wherein the first powder contains an indacaterol salt and mometasone furoate as active ingredients.

7. A formulation according to claim 4, wherein the first powder contains an indacaterol salt, glycopyrrolate and mometasone furoate as active ingredients.

8. A formulation according to claim 1, wherein the first spray-dried powder and a second spray-dried powder contain the same hydrophobic excipient.

9. A formulation according to claim 8, wherein the hydrophobic excipient is a phospholipid.

10. A formulation according to claim 1, wherein a fine particle dose less than 3.3 μm is greater than 40% to minimize interpatient variability associated with oropharyngeal deposition.

11. A formulation according to claim 1, wherein a fine particle dose less than 4.7 μm is greater than 50% to minimise interpatient variability associated with oropharyngeal deposition.

12. A formulation according to claim 1, wherein a variability in the fraction of particles with a d²Q<500 (expressed as the mean variability) is less than 20% across a range of pressure drops in a dry powder inhaler from 2 kPa to 6 kPa.

13. The powder formulation of claim 1, and further including a receptacle for inhalation wherein the receptacle comprises a fill mass of from 0.5 mg to 10 mg.

14. The powder formulation of claim 13 wherein the active has a crystallinity content of at least 90%.

15. The powder formulation of claim 13 wherein the crystalline therapeutically active ingredient has a solubility of between 0.1 and 1.0 mg/ml.

16. A process for preparing an inhalable dry powder formulation of spray-dried particles, the process comprising the steps of:
(a) preparing a first feedstock comprising a crystalline active ingredient dispersed in a liquid phase and a hydrophobic excipient dispersed or dissolved in a liquid phase and spray-drying said first feedstock to provide a first engineered dry powder, wherein a drug loading of the crystalline active agent results in less than 10% w/w active dissolution in the solvent phase of the feedstock;
(b) preparing a second feedstock comprising a hydrophobic excipient dissolved or dispersed in a liquid phase, said second feedstock being substantially free of the active ingredient, and spray-drying said second feedstock to provide a second engineered dry powder substantially free of active ingredient; and
(c) mixing the active dry powder particles and the non-active dry powder particles to provide an inhalable dry powder formulation.

17. A process according to claim 16 wherein a proportion of the active dry powder particles and the non-active dry powder particles is adjusted to deliver a target dose of active ingredient.

18. A process according to claim 16 where the contents of the drug content of the first feedstock is such that the drug content of the active dry powder particles formulation is sufficient to achieve the desired drug content of the inhalable dry powder formulation.

19. A process according to claim 16 where the active dry powder particles and the non-active dry powder particles are mixed prepared and mixed substantially simultaneously.

20. A process according to claim 16 where the solubility of the active ingredient in the solvent phase of the first feedstock is between 0.1 g/ml and 2 g/ml.

21. A process according to claim 16 where a percentage dissolved active ingredient in the feedstock is less than 5% w/w.

22. A process according to claim 16 wherein the first feedstock and the second feedstock are passed through a twin-fluid atomiser that spray dries the feedstocks and mixes the active dry powder particles and non-active dry powder particles that are prepared from the respective feedstocks to give the inhalable dry powder formulation.

23. A process according to claim 22 wherein the twin-atomiser comprises from two to six independently controllable twin-fluid nozzles.

24. A process according to claim 22 wherein the first feedstock and the second feedstock are passed through separate atomisers that spray dry the feedstocks.

25. A method for the treatment of an obstructive or inflammatory airways disease which comprises administering to a subject in need thereof an effective amount of a dry powder formulation according to claim 1.

26. (canceled)

27. (canceled)

28. A delivery system, comprising an inhaler and a dry powder formulation for inhalation according to claim 1.
